(19) United States
(12) Patent Application Publication
(10) Pub. No.: US 2023/0152320 A1
(43) Pub. Date: May 18, 2023

(54) APPARATUS AND METHOD FOR AIRBORNE PATHOGEN DETECTION USING AN ELECTROCHEMICAL PLATFORM

(71) Applicants: University Of Maryland, Baltimore, Baltimore, MD (US); University of Maryland, Baltimore County, Baltimore, MD (US)

(72) Inventors: Dipanjan PAN, State College, PA (US); Maha Mohammad Shehadeh ALAFEEF, State College, PA (US)

(21) Appl. No.: 18/055,333

(22) Filed: Nov. 14, 2022

Related U.S. Application Data
(60) Provisional application No. 63/279,360, filed on Nov. 15, 2021.

(51) Int. Cl. G01N 33/569; G01N 29/12; G01N 29/24; G01N 33/543

(52) U.S. Cl. CPC G01N 33/56983; G01N 29/12; G01N 29/2443; G01N 33/54373; G01N 2291/014; G01N 2291/023; G01N 2333/165

(57) ABSTRACT

An apparatus is provided for airborne pathogen detection, which includes a crystal microbalance. The apparatus includes specific capture probes that are affixed to the crystal microbalance and are designed to bind to and capture a specific pathogen, such as a virus particle. This capture causes a change in mass of the crystal microbalance that can be detected. A method is provided for airborne pathogen detection, which includes calibrating a resonant frequency of the crystal microbalance to a mass on the crystal microbalance. The method also includes a step of conjugating the antibody to the crystal microbalance. The method also includes, for each measurement time, measuring a resonant frequency of the crystal microbalance and determining a mass change due to binding of the pathogen to the detector. This mass change is then related to pathogen load in the medium. A notification is output if the viral load exceeds a predetermined threshold.

### CROSS-REFERENCE TO RELATED APPLICATIONS

This application claims benefit of Provisional Appln. 63/279,360, filed Nov. 15, 2021, the entire contents of which are hereby incorporated by reference as if fully set forth herein, under 35 U.S.C. § 119(e).

### BACKGROUND

Airborne respiratory diseases (e.g., COVID-19) caused by novel viruses (e.g. SARS-CoV-2) have recently been identified as causative of a wide range of clinical outcomes. Recent outbreak of such novel viruses has been declared pandemics by the World Health Organization (WHO). Many tests have been authorized by the Food and Drug Administration (FDA) to identify cases of such airborne respiratory diseases, in efforts to slow its spread.

Slowing the infection rate of an infection disease helps to minimize overwhelming of overworked healthcare facilities, thereby allowing for more effective treatment of existing cases and delaying new cases before therapeutics or a vaccine is available. However, these tests have notable drawbacks. For example, regular screening of the population in high-risk areas using tests levies a financial burden on the healthcare system. Further, most of these tests are not sensitive enough to detect the presence of the virus in its early stages and require a substantial viral load for accurate detection. In addition, most of these tests are not sensitive enough to detect the presence of the virus in its early stages and require a substantial viral load for accurate detection. There is a need in the art for methods and systems for detection of viruses and other pathogens in the environment, for example the ambient air, in a particular location.

### SUMMARY

Therefore, techniques are provided here for airborne pathogen detection using an electrochemical platform. The inventors of the present invention developed an apparatus and method for airborne pathogen detection using an electrochemical platform which addresses the drawbacks of conventional tests. To address these drawbacks, provided is a more reliable, efficient, and cost-effective pathogen detection device configured to be used in areas with high footfall (e.g., shelters, healthcare facilities, and dormitories). The device can increase the likelihood of detecting the presence of a pathogen (e.g., as compared with individual testing) and provide authorities with data identifying particular areas as potential sources of infection. Thus, the device can be used to help identify high-risk zones and implement measures to identify and quarantine those with recent visits to particular high-risk zones to reduce the likelihood of further spread of a particular infectious disease.

In a first set of embodiments, an apparatus is provided. The apparatus includes a crystal microbalance (preferably a quartz crystal microbalance (QCM)) with one or more types of specific capture probes 250 adsorbed or attached to the (preferably gold) electrode on its surface or the surface of a capture membrane 720, which is adjacent to and in contact with the electrode on one side of the crystal microbalance in some embodiments. This capture membrane can help in improving the system specificity and consists of a two dimensional material such as molecularly imprinted polymers (MIPs), a covalent organic framework, or a metal-organic framework (MOF), and the like, with a layer of capture probes, and is designed to capture the airborne target from the air. The capture membrane is optional. The capture probes 250 include a first region which is conjugated to the electrode of the crystal microbalance or the capture membrane 720, and a second region that specifically binds to a pathogen or portion thereof, for example a virus particle, to cause a change in mass on the crystal microbalance which correlates to the amount of pathogen, and can be detected.

In a second set of embodiments, a method is provided including a step of providing a crystal microbalance configured to detect a change in mass. The method also includes a step of providing a plurality of one or more specific capture probes 250 that are capable of being adsorbed onto or conjugated to the surface of the crystal microbalance 201 electrode or capture membrane and that also specifically binds to a pathogen or a portion thereof. This capture probe 250 is prepared and designed to bind to a particular pathogen or set of pathogens and is absorbed onto or bound onto the crystal microbalance 201 so that capture of the pathogen (for example a virus particle) results in a change in mass to be detected by the crystal microbalance as described below. The capture probe 250 is conjugated to the crystal microbalance 201 as discussed herein below.

In a third set of embodiments, a system is provided including a crystal microbalance according to the first set of embodiments. The system also includes an oscillator connected to a pair of electrodes and configured to generate an alternating current (AC) voltage across a crystal of the crystal microbalance to cause the crystal to vibrate at a resonant frequency. The system also includes a frequency detection unit configured to measure a shift in the resonant frequency due to the change in the mass caused by the pathogen binding specifically to the probe(s) 250 in a medium directed at the crystal microbalance. The system also includes a processor and a memory including one or more sequences of instructions. The memory and the sequences of instructions are configured to, with the processor, cause the system to transmit a first signal to the oscillator to cause the oscillator to generate the AC voltage across the crystal; to receive a second signal from the frequency detection unit indicating a value of the shift in the resonant frequency due to the change in the mass; to determine a mass change of the crystal microbalance based on the shift in the resonant frequency; to relate the mass change to a pathogen load in the medium; to determine whether the pathogen load exceeds a high load threshold and to output a notification indicating an excessive load in the medium based on determining that the pathogen load exceeds the high load threshold.

In a fourth set of embodiments, a method is provided including a step of calibrating a resonant frequency of the crystal microbalance according to the first set of embodiments to a mass on the crystal microbalance. The method also includes a step of conjugating the specific capture probe(s) 250 to the crystal microbalance electrode 204 or capture membrane 720 in a sensor chip. The method also includes installing the sensor chip in a housing and directing a medium at the sensor chip. The method also includes, for each measurement time, measuring a resonant frequency of the crystal microbalance and determining a mass change based on the measured resonant frequency. The method also includes relating the mass change to a pathogen load in the medium. The method also includes determining whether the pathogen load exceeds a high load threshold. The method also includes outputting a notification indicating an excessive load in the medium based on determining that the pathogen load exceeds the high load threshold.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is an image that illustrates an example of a quartz crystal microbalance (QCM), according to an embodiment;

FIG. 1B and FIG. 1C are images that illustrate an example of the crystal of the quartz crystal microbalance of FIG. 1A undergoing oscillation due to an applied AC voltage, according to an embodiment;

FIG. 1D is a graph that illustrates an example of a shift in a resonant frequency of the QCM of FIG. 1A based on increasing mass on the QCM, according to an embodiment;

FIG. 1E is a circuit diagram that illustrates an example of a circuit representation of the QCM of FIG. 1A, according to an embodiment;

FIG. 1F is a graph that illustrates an example of an impedance measured across the electrodes of FIG. 1A for a range of oscillation frequencies of the QCM, according to an embodiment;

FIG. 2A is a block diagram that illustrates example of a virus detection system using the QCM of FIG. 1A, according to an embodiment;

FIG. 2B is a block diagram that illustrates an example of a specific capture probe conjugated to a surface of the QCM and bound to a pathogen in a medium directed at the QCM, according to an embodiment;

FIG. 2C is a block diagram that illustrates an example of an antibody specific capture probe conjugated to a surface of the QCM and bound to a virus particle in a medium directed at the QCM, according to an embodiment;

FIG. 2D is an image that illustrates an example of the QCM of the system of FIG. 2A, according to an embodiment;

FIGS. 3A through 3G are graphs that illustrate an example of data obtained during a calibration of the systems, according to an embodiment;

FIG. 4 is a flow diagram that illustrates an example method to detect a viral load in a medium with the system of FIG. 2A, according to an embodiment;

FIGS. 5A and 5B are images that illustrates example of a viral detection system including a housing to direct a medium at the QCM of the system, according to an embodiment;

FIGS. 6A through 6C are images that illustrates example of a viral detection system including a housing to direct a medium at the QCM of the system, according to an embodiment;

FIGS. 7A and 7B are images that illustrates example of a viral detection system including a housing to direct a medium at the QCM of the system, according to an embodiment;

FIGS. 8A through 8C are images that illustrates example of a viral detection system including a housing to direct a medium at the QCM of the system, according to an embodiment;

FIG. 9 is a block diagram that illustrates a computer system upon which an embodiment of the invention may be implemented; and

FIG. 10 illustrates a chip set upon which an embodiment of the invention may be implemented.

### DETAILED DESCRIPTION

A method and apparatus are described for airborne pathogen (e.g., virus) detection using an electrochemical platform. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

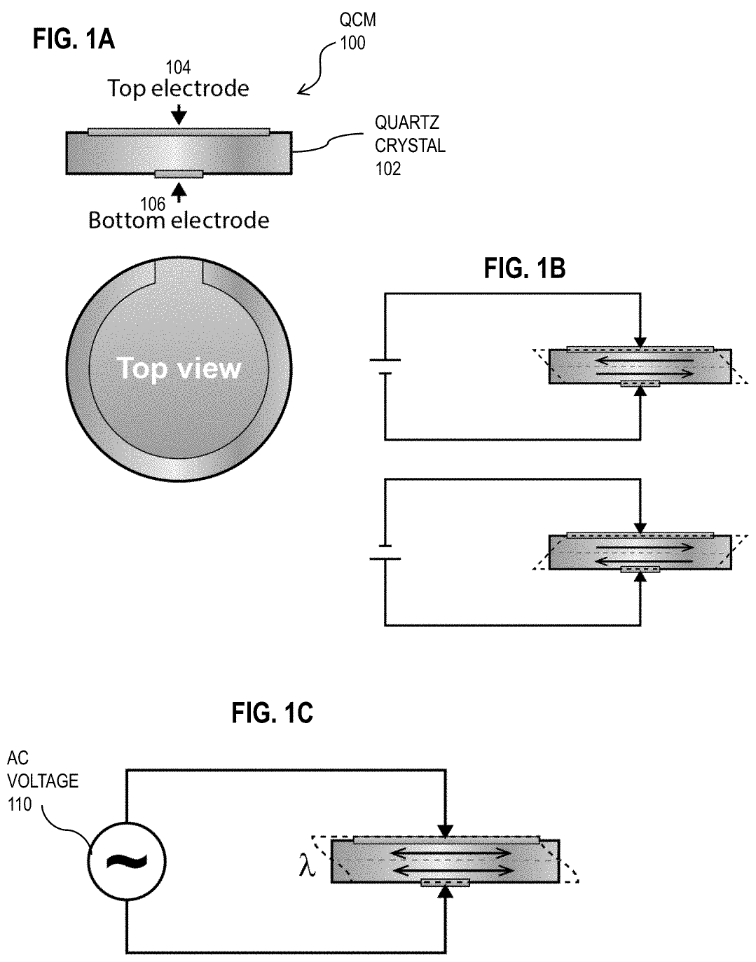

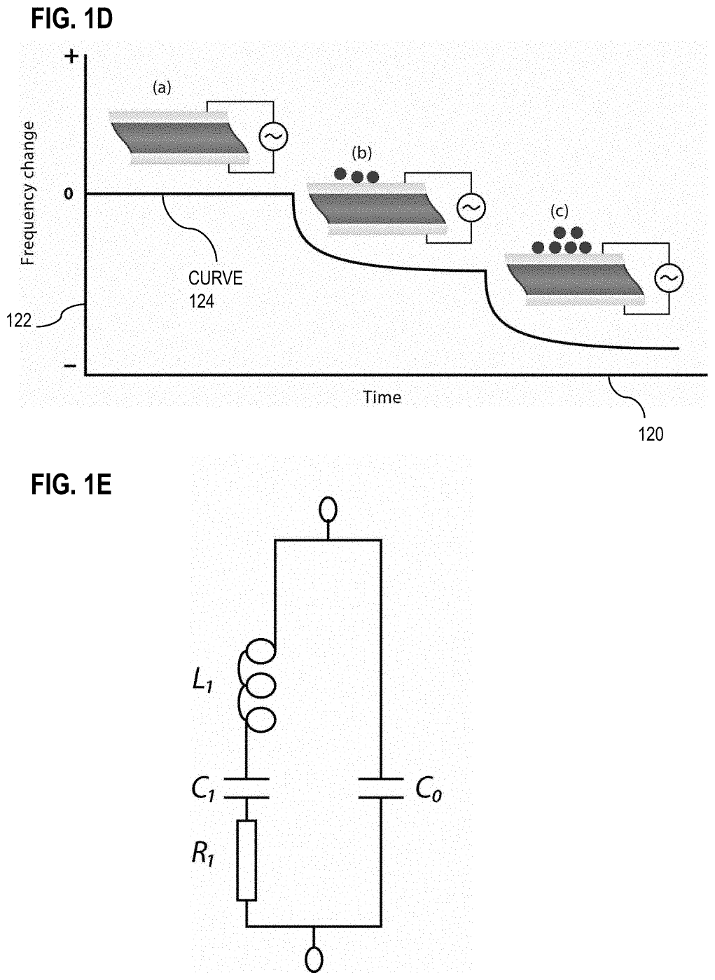

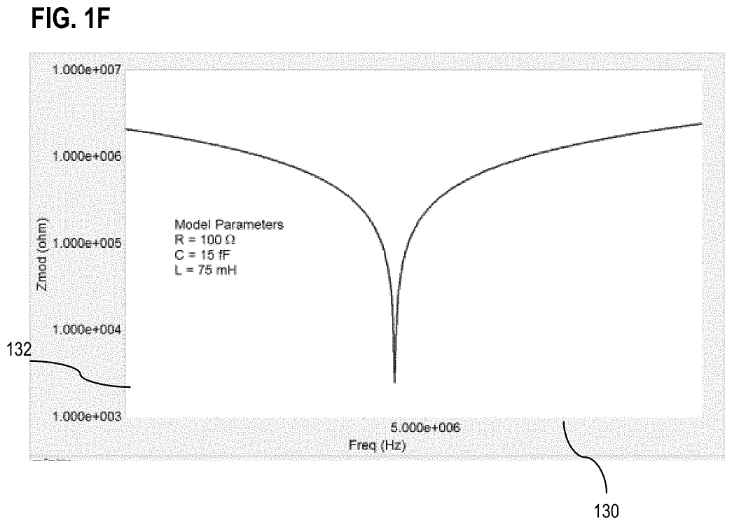

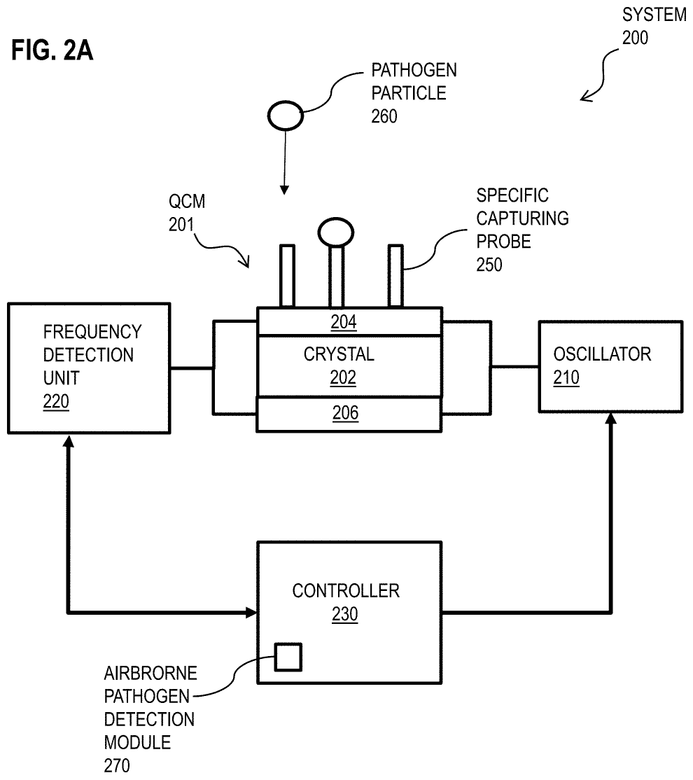

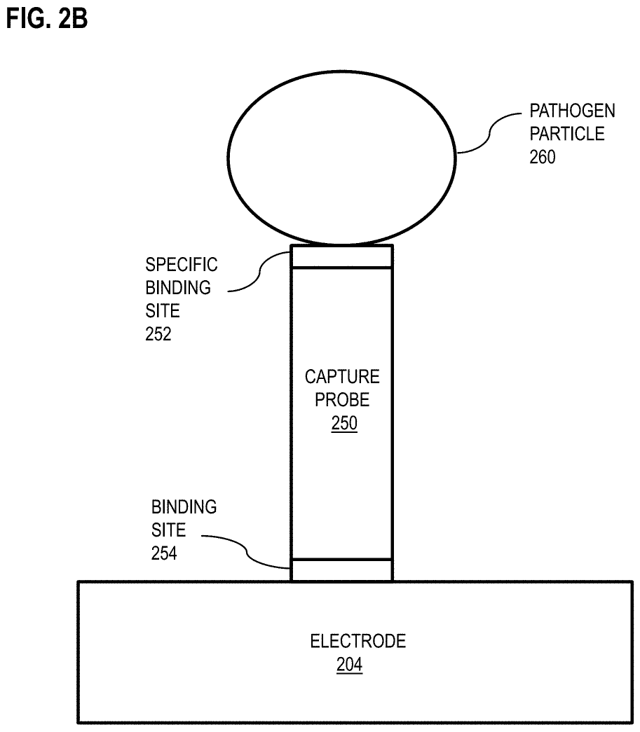

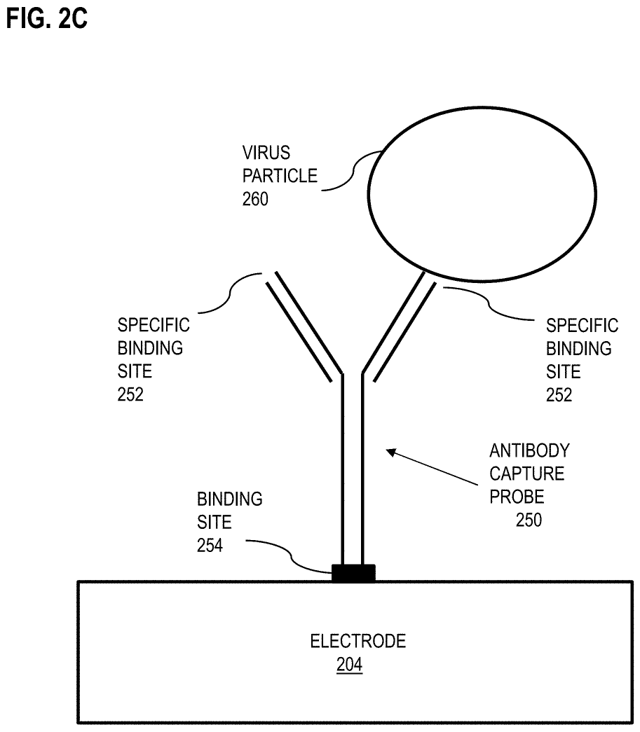

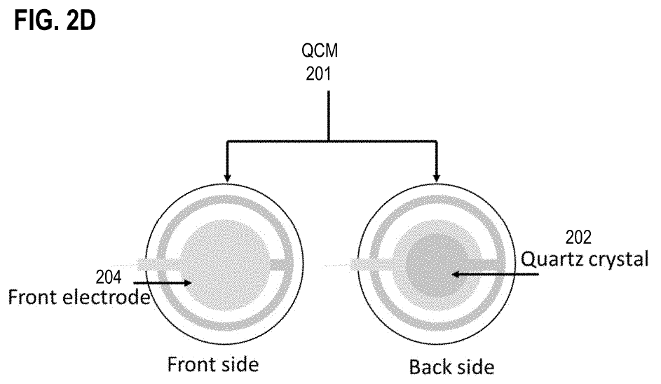

As used herein, the term “crystal microbalance” refers to an apparatus comprising an acentric crystalline, piezoelectric material, preferably a quartz crystal, cut from a bulk crystal at specific orientations with respect to the crystallographic axis and directly evaporated onto the top and bottom of this cut crystal, a pair of metal electrodes (usually gold). See FIG. 1A and FIG. 2D.

Specific capture probes are fixed onto the top electrode of the crystal microbalance, allowing the microbalance to capture specific material to be detected by mass added to the microbalance.

Some embodiments of the invention are described below in the context of a quartz crystal microbalance (QCM) used to directly detect virus particles in a dry state from an air sample. The device directs the air from the inlet to the surface of the electrode where the airborne pathogen target interacts with the capturing probes to produce a detectable signal. The device is not only useful in detecting respiratory viruses, but also can be used to detect bacterial contamination and fungi. In addition, it can be helpful in air quality control monitoring. However, the invention is not limited to this context.

### 1. QUARTZ CRYSTAL MICROBALANCE (QCM) OVERVIEW

Quartz Crystal Microbalance (QCM) is an extremely sensitive mass balance that measures nanogram to microgram level changes in mass per unit area. The heart of the technology is a quartz crystal. Quartz is a piezoelectric material that can be made to oscillate at a defined frequency by applying an appropriate voltage, usually via metal electrodes. The frequency of oscillation can be affected by the addition or removal of small amounts of mass onto the electrode surface. This change in frequency can be monitored in real time to obtain useful information about the addition or removal of the small amounts of mass on the electrode surface.

The change in mass on the quartz crystal is linearly related to the change in the oscillation frequency of the quartz crystal which is defined by a well-known equation called the Sauerbrey Equation. The QCM can provide useful information on the amount of mass deposited and the rate of deposition (or removal) on the quartz crystal by monitoring the real-time change in frequency [1]-[2].

The operation of QCM is based on the piezoelectric effect that occurs in crystalline materials of certain crystallography known as “acentric” materials [3]-[4]. Quartz belongs to this class of crystals. The word piezoelectricity is derived from the Greek word “piezein”, which means “to press”, and the electricity that is generated in response to applied pressure in these types of materials. Piezoelectricity is defined as the generation of electricity in response to the mechanical deformation caused by mechanical stress or as the generation of physical deformation on the application of electricity in such crystals. The French physicists Pierre and Jacques Curie discovered this effect in 1880 when they demonstrated that salt crystals could produce electricity when deformed along certain crystallographic orientations [3]. A year later they demonstrated that the converse effect was also possible, i.e., the quartz could deform upon the application of voltage.

Quartz, besides being piezoelectric, also possesses a unique combination of properties that make it an ideal candidate for ultrasensitive devices. It is found in abundance in nature, and it is easy to grow and process. In addition, α-quartz, the phase of quartz that can be used as a resonator, is thermodynamically stable up to 573° C.

To fabricate quartz crystal resonators, wafers are cut from a bulk quartz crystal at specific orientations with respect to the crystallographic axis. The quartz crystals used in QCMs are most commonly processed using the “AT cut” that provides pure thickness shear mode oscillation where the two surfaces of the crystal move in an anti-parallel fashion [5].

FIG. 1A shows an example of a quartz crystal microbalance (QCM) 100 that includes a quartz crystal 102. After cutting the quartz crystal, a pair of metal electrodes 104, 106 (usually gold) is directly evaporated on the top and bottom surfaces of the quartz crystal 102. As further shown in FIGS. 1B and 1C, when an alternating current (AC) voltage 110 is applied to the quartz crystal 102, it oscillates. The arrows in FIGS. 1B and 1C indicate the direction of a shear wave oscillation generated in the quartz crystal 102 due to the applied AC voltage 110. The resonance frequencies of these quartz crystals are typically on the order of MHz and inversely proportional to the crystal thickness. The common 5 MHz quartz crystal has a corresponding thickness of approximately 330 μm. The application of the AC voltage 110 (a sine wave in nearly all cases) to the crystal faces causes the crystal to oscillate. When the thickness of the crystal (t_{q}) is twice the acoustical wavelength, a standing wave can be established where the inverse of the frequency of the applied potential is of the period of the standing wave. This frequency is called the resonant frequency, f₀, and is given by the equation:

$${f}_{0} = \frac{\sqrt{\frac{{μ}_{q}}{{ρ}_{q}}}}{2 {t}_{q}} \qquad ( 1 )$$

where μ_{q} is the shear modulus (a ratio of sheer stress to shear strain), ρ_{q} is the density, and t_{q} is the crystal thickness. The amount of energy lost during oscillation at this frequency is at a minimum.

The frequency change in QCMs can be measured with a resolution of 1 Hz or less on crystals with a fundamental resonance frequency in the MHz range. Because of its high stability as a resonator, quartz crystals were successfully incorporated, in the early 1900s, as components in various devices such as electronic filters, frequency control devices, and ultrasonic transducers. The application of quartz crystals as sensitive mass balances was realized in the late 1950s following the pioneering work of Sauerbrey. Sauerbrey demonstrated, in 1959, that the frequency change (Δf) of an oscillating quartz crystal 102 could be linearly related to its mass change (Δm) as expressed by:

$$Δ m = \frac{- C \times Δ f}{n} \qquad ( 2 )$$

where n is the overtone number and C is a constant that depends on the property of the crystal used. Equation 2, typically referred to as the Sauerbrey equation, constitutes the basic principle of QCM technology [2]. For a 5 MHz AT-cut quartz crystal at room temperature, C is approximately equal to 17.7 ng/(cm²·Hz). This means that the addition of 17.7 ng/cm² of mass on a 5 MHz quartz crystal causes a frequency change of 1 Hz. The frequency of 5 MHz quartz can be easily measured with a precision of 0.01 Hz in a vacuum; therefore, measurement of nanogram-scale masses can be achieved. For example, the corresponding frequency shift on the addition of a monolayer of water with an areal density of approximately 25 ng/cm² to the surface of an AT-cut quartz crystal is approximately 1.4 Hz and well within the limits of detection.

Sauerbrey developed Equation 2 assuming that a small mass added to the crystal can be treated as an equivalent change in the mass of the quartz crystal itself. This means that the equation is only valid when the added mass is rigidly adsorbed on the quartz crystal surface with no slip. Sauerbrey's finding is schematically depicted in FIG. 1D which shows that the resonance frequency decreases as more mass is rigidly absorbed on the quartz crystal surface. The horizontal axis 120 in FIG. 1D is time (arbitrary units), the vertical axis 122 in FIG. 1D is a shift in the resonant frequency of the quartz crystal 102 (arbitrary units). The curve 124 in FIG. 1D indicates a value of a shift in the resonant frequency of the quartz crystal 102 based on the addition of mass to the quartz crystal 102. Note that as the amount of mass on the quartz crystal 102 increases (from (a) to (c) in FIG. 1D) the value of the resonant frequency of the quartz crystal 102 decreases. At a certain point, when the value of the resonant frequency has reached too low of a value and/or the surface of the quartz crystal 102 is full of added mass, the quartz crystal 102 can no longer detect the addition of more mass. In this instance, the added mass is removed from the quartz crystal 102 which causes the resonant frequency value to return to an initial value. At this point, the quartz crystal 102 can be used again to detect an addition of mass.

The detection of the shift in the frequency of the quartz crystal is now discussed. The quartz crystal 102 of FIG. 1A can be represented by a mechanical circuit model (FIG. 1E) [6]-[7]. Specifically, the quartz crystal 102 (and any added mass) is represented by the “RLC” portion of the left side of the circuit whereas the electrodes 104, 106 are represented by the capacitance C₀ on the right side of FIG. 1E. This circuit is commonly referred to as the Butterworth van Dyke (BvD) model [8]. The electrical circuit model consists of inductance, L₁, capacitance, C₁, and resistance, R₁, in series, and a parallel shunt capacitance, C₀. Here R₁ represents the energy dissipated during oscillation, C₁ represents the energy stored during oscillation, and L₁ represents the inertial component related to the displaced mass. At the resonant frequency, f₀, the impedance of the circuit is at a minimum and is equal in magnitude to R₁ as shown in FIG. 1F [8]. The horizontal axis 130 in FIG. 1F is frequency (arbitrary units) and the vertical axis 130 in FIG. 1F is impedance (arbitrary units).

Thus, to determine a shift in a resonant frequency due to a change in mass on the quartz crystal 102, the impedance is measured across the electrodes 104, 106 over a range of frequencies after this change in mass. The value of the impedance is a minimum at the resonant frequency. This value of the resonant frequency is subtracted from a previous known value of the resonant frequency (prior to the addition of the mass) to determine the shift in the resonant frequency. This shift in the resonant frequency is determined by a frequency detection unit which is connected across the electrodes 104, 106. The AC voltage 110 is applied which causes the quartz crystal 102 to vibrate at a range of frequencies including the natural frequency (e.g. 5 MHz) of the quartz crystal 102. Over the range of frequencies, the frequency detection unit measures the value of the impedance and determines the value of the frequency at which the impedance is a minimum value. The frequency detection unit then determines the shift in the resonant frequency by comparing this value with a previous known value of the resonant frequency (prior to adding the mass). In another example, instead of measuring the impedance value over the range of oscillation frequencies, the frequency detection unit measures an admittance (inverse of the impedance) and determines the value of the resonant frequency based on the frequency value where the admittance has a maximum value [9].

### 2. OVERVIEW

A system will now be discussed that includes the QCM of FIG. 1A and is used to detect a pathogen load (e.g., viral load) in a medium (e.g. air, water, etc.). To address the limitations of conventional virus testing, the inventors of the present invention developed a more reliable, efficient, and cost-effective viral detection device configured in areas with high footfall like shelters, healthcare facilities, and dormitories (compared to typical devices). The device can increase the likelihood of detecting the presence of a virus (e.g., such as with individual testing) and provide authorities with data to designate particular areas with presence of the virus. Thus, the device can be used to help identify high-risk zones and implement measures to identify and quarantine those with recent visits to particular high-risk zones to reduce the likelihood of further spread of a virus. FIG. 2A is a block diagram that illustrates example of a pathogen detection system 200 using the QCM 100 of FIG. 1A, according to an embodiment. The system 200 includes a QCM 201 that is similar to the QCM 100 previously discussed, with the exception of the features discussed herein.

In an embodiment, the QCM 201 includes one or more specific pathogen probes 250. In an example embodiment, the specific pathogen probes 250 are conjugated to the electrode 204 of the QCM 201 or the capture membrane 720. Additionally, the specific pathogen probes 250 are configured to bind to one or more pathogens 260 in the air (or in a medium directed at the QCM 201). As each pathogen 260 binds to the pathogen probes 250, a mass on the QCM 201 increases, which in turn causes a shift in the resonant frequency of the QCM 201. The system 200 is configured to detect this shift in the resonant frequency of the QCM 201 and then determine a change in mass on the QCM 201 based on this resonant frequency shift. In one embodiment, the system 200 then assesses a pathogen load attributable to this mass change (e.g. determines how many pathogen particles 260 were bound to the QCM 201 via the capture probes in a given time). In this embodiment, the system 200 also determines whether to issue an alert notification based on whether the viral load exceeds a high load threshold.

An AC voltage is applied across the crystal 202 of the QCM 201 to induce oscillation of the crystal 202. In an embodiment, the system 200 includes an oscillator 210 that is connected to the electrodes 204, 206 of the QCM 201. In one embodiment, the oscillator 210 is configured to apply an AC voltage across the electrodes 204, 206. In an example embodiment, the AC voltage is similar to the AC voltage 110 of FIG. 1C which causes the crystal 202 to oscillate at a range of frequencies including a natural frequency (e.g. about 5 or 10 MHz). A quartz crystal oscillator driver can be also used to power the QCM to reduce the power consumption and increase the quartz crystal frequency stability. The oscillator driver can be biased using a DC source as low as 3.3 V supply voltage. In an embodiment, the oscillator 210 continuously applies the AC voltage across the electrodes 204, 206 while the system 200 is in an active mode (e.g. to detect a pathogen load in the air based on sensing a change in mass on the QCM 201).

The resonant frequency of the crystal 202 is determined as the crystal 202 oscillates due to the applied AC voltage. In an embodiment, the system 200 includes a frequency detection unit 220 that is connected to the electrodes 204, 206 of the QCM 201. In one embodiment, the frequency detection unit is configured to measure an impedance across the electrodes 204, 206 for a range of crystal oscillation frequencies (as the AC voltage is applied). The frequency at which the measured impedance is a minimum value is the resonant frequency of the crystal 202. In other embodiments, the frequency detection unit 220 measures an admittance (inverse of the impedance) across the electrodes 204, 206 for a range of crystal oscillation frequencies. In these embodiments, the frequency at which the admittance is a maximum value is the resonant frequency of the crystal 202. In an embodiment, the frequency detection unit 220 is configured to determine the resonant frequency of the crystal 202 at different measurement times and measured impedance is at a minimum value for the resonant frequency of the apply an AC voltage across the electrodes 204, 206. In an embodiment, the frequency detection unit 220 determines the resonant frequency of the crystal 202 over one or more measurement time periods (during which a pathogen load in a medium incident on the QCM 201 is sought to be measured).

The system 200 is operated using a controller 230 which transmits and receives one or more signals. As shown in FIG. 2A, in one embodiment the controller 230 is communicatively coupled with the oscillator 210 and the frequency detection unit 220. In an embodiment, the controller 230 transmits a first signal to the oscillator 210 to commence application of the AC voltage across the electrodes 204, 206 to induce oscillation of the crystal 202. Additionally, in an embodiment the controller 230 transmits a second signal to the frequency detection unit 220 to detect the resonant frequency of the crystal 202 due to oscillation after applying the AC voltage. Additionally, as shown in FIG. 2A in some embodiments, the frequency detection unit 220 transmits signals to the controller 230. In an example embodiment, the frequency detection unit 220 transmits a signal to the controller 230 that indicates the resonant frequency of the crystal 202. In other embodiments, the frequency detection unit 220 transmits data to the controller 230 of the measured impedance at the range of oscillation frequencies and the controller 230 determines the resonant frequency based on that frequency with the minimum impedance value.

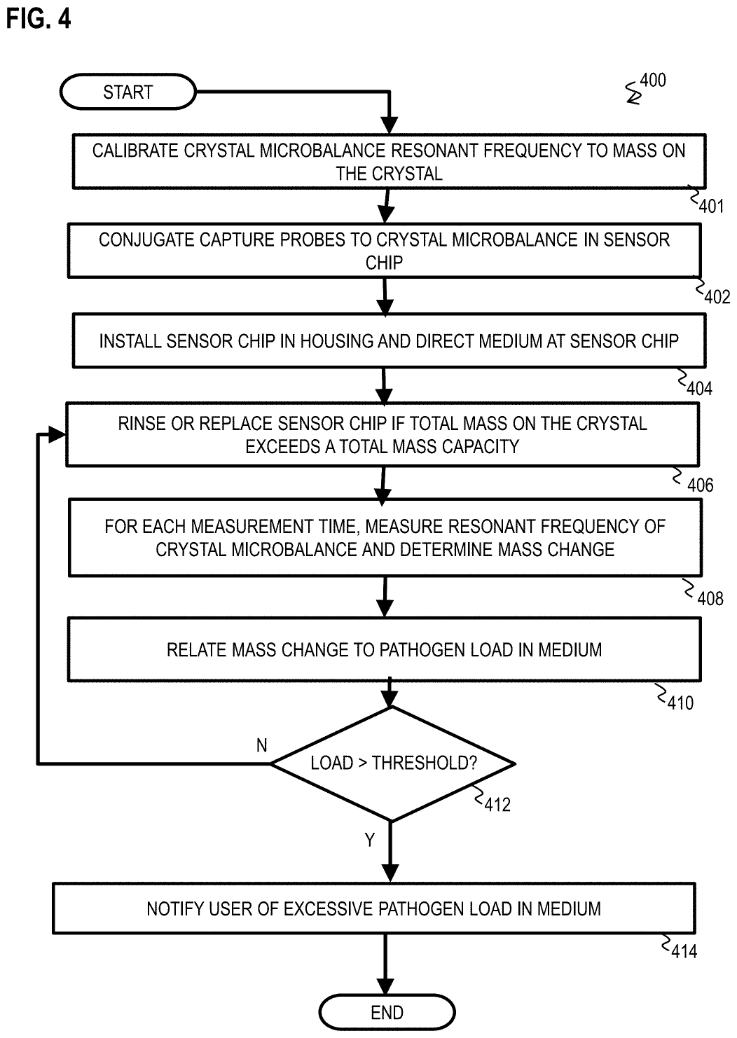

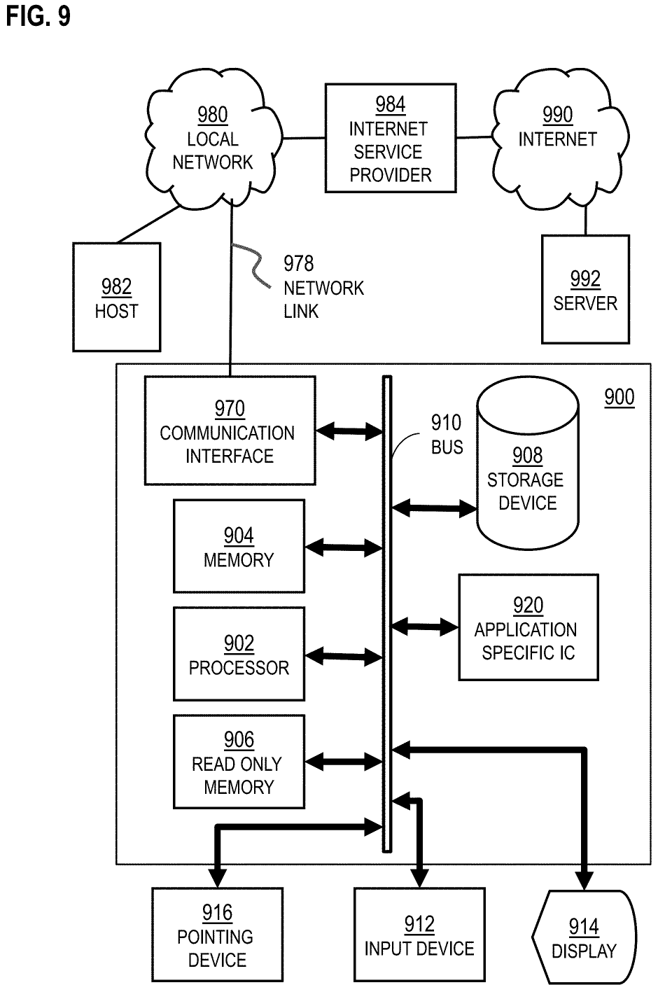

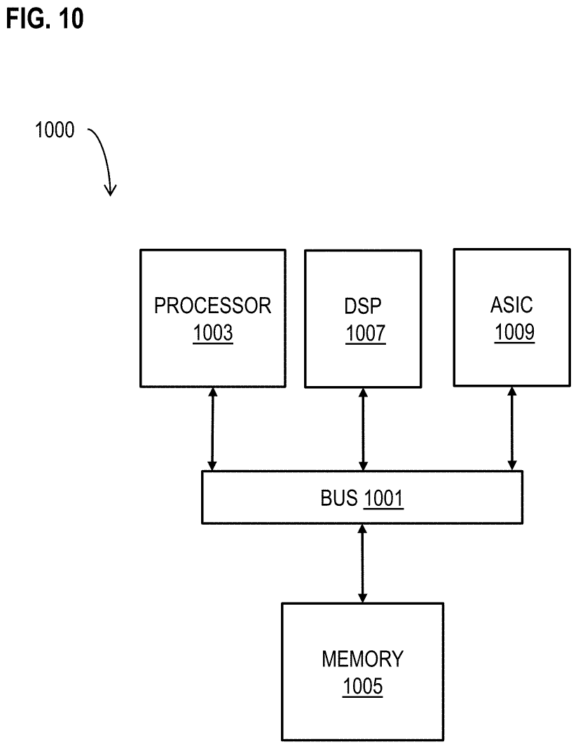

In various embodiments, the controller 230 includes an airborne pathogen detection module 270 that includes instructions to cause the controller 230 to perform one or more steps of the method 400 of FIG. 4. In still other embodiments, the controller 230 is a general purpose computer system, as depicted in FIG. 9 or one or more chip sets as depicted in FIG. 10.

The specific capture probe 250 of the system 200 is now discussed in more detail. FIG. 2B and FIG. 2C are block diagrams that illustrates examples of the capture probe 250 conjugated to a surface of the QCM 201 and bound to a pathogen particle 260 in a medium directed at the QCM 201, according to an embodiment. In one embodiment, the capture probe 250 is attached to the QCM 201 (e.g. they are integrated in one chip). In another embodiment, the capture probe 250 is attached to the electrode 204 (e.g. gold electrode). In some embodiments, the capture probe 250 is optional and is used to help in increasing the specificity of the pathogen particle 260 whose load is being detected by the system 200. The probe can be covalently linked to the gold or any other type (e.g. silver, platinum, etc.) of metal electrode using a chemical linker as known in the art. In an embodiment, the capture probe 250 includes a first portion or binding site 254 which conjugates the antibody 250 to the electrode 204. In an embodiment, the capture probe 250 is an antibody and includes a binding region 254 for conjugation to the electrode surface and at least one specific antibody binding region for binding to a pathogen particle.

Suitable specific capture probes for embodiments of the invention include any natural or artificial binding molecule known in the art that is capable of specifically binding to a pathogen or portion thereof and also is capable of binding to, adsorbing to, or being conjugated to the surface of the microbalance electrode. In particular, suitable capture probes include, but are not limited to, peptides, proteins, and oligonucleotides. Preferably, the capture probe is an antibody or aptamer, and most preferably is a monoclonal antibody. Such specific binding probes are known in the art, as well as methods for making and testing them.

Proteins and peptides are preferred capture probes, however oligonucleotides that specifically bind to a pathogen, such as a pathogen particle, or portion thereof can be used as a capture probe. The most preferred capture probe is an antibody or population of antibodies, including polyclonal and monoclonal antibodies of any class. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity. The most highly preferred specific capture probe according to embodiments of the invention is a high-affinity monoclonal antibody that specifically binds the S-protein of SARS-CoV-2, the causative agent of COVID19.

Antibody fragments known in the art are also included in the definition of antibody as used herein. “Antibody fragments” comprise a portion of an intact antibody comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments. Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to tissues. For a review of certain antibody fragments, see Hudson et al. (2003) Nat. Med. 9:129-134, which is hereby incorporated by reference in its entirety.

Traditionally, antibody fragments can be derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10: 163-167 (1992)). According to another approach, F(ab′)2 fragments can be isolated directly from recombinant host cell culture. Fab and F(ab′)2 fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific. All of the references contained in this paragraph are hereby incorporated by reference in their entirety.

Antibody variants also are included in the definition of antibody in the present application. Thus, in some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made and glycosylation of the protein sequence can be added, deleted or modified. Methods for accomplishing these modifications are well known and understood by those of skill in the art.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

Aptamers are nucleic acid or peptide molecules that bind to a particular molecule of interest with high affinity and specificity (Tuerk and Gold, Science 249:505 (1990); Ellington and Szostak, Nature 346:818 (1990)), and are suitable for use with embodiments of the invention as specific capture probes. DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000). Aptamers may be RNA or DNA based and may include a riboswitch. Regulatory elements are known as riboswitches and are defined as mRNA elements that bind metabolites or metal ions as ligands and regulate mRNA expression by forming alternative structures in response to this ligand binding (FIG. 1; Nudler & Mironov 2004; Tucker & Breaker 2005; Winkler 2005). Although they can bind proteins like antibodies, aptamers are not immunogenic, even at doses up to 1000 times the therapeutic dose in primates. Aptamers are suitable for use in embodiments of the invention.

“Binding affinity” generally refers to the strength of the sum total of noncovalent interactions between a single (specific) binding site of a molecule (e.g., an antibody) and its binding partner (e.g., an antigen, or according to embodiments of the invention, a pathogen or a virus particle). High-affinity antibodies generally bind antigen faster and tend to remain bound longer, and have a Kd of less than about 10⁻⁶ molar; more preferably less than about 10⁻⁷ molar, less than about 10⁻⁸ molar or less than about 10⁻⁹ molar. According to embodiments of the invention, capture probes with high affinity are preferred.

The QCM 201 of the system 200 is similar to the QCM 100 of FIG. 1A. FIG. 2D is an image that illustrates an example of the QCM 201 of the system 200 of FIG. 2A, according to an embodiment. In an embodiment, as shown in FIG. 2D the electrode 204 has a key-hole shape. In an example embodiment, an antibody or other capture probe 250 is conjugated or bound to a portion of the electrode 204 that overlaps with the quartz crystal 202 and does not extend beyond this overlap region between the electrode 204 and quartz crystal 202.

In summary, any molecule can be used as a capture probe, and such capture probes are produced or engineered to contain a specific binding site of choice for a pathogen to be assayed and a binding site that can be conjugated to the electrode surface on the crystal microbalance. Production of proteins, peptides, aptamers, and other biomolecules that specifically bind to a structure with high affinity are well known in the art and can readily be produced by persons of skill. The specific capture probes can be conjugated to a surface using any of the known methods in the art.

The binding site 254 of a protein or peptide capture probe such as an antibody can be conjugated to the gold surface using any known method. For example, the formation of a self-assembled monolayer can be used to link the probe to the surface using EDC/NHS chemistry. The use of chemical linkers such as 3,3′-dithiobis(sulfosuccinimidyl propionate) in the presence of reducing agent allow the direct linking of the probe to the electrode surface.

The specific binding site(s) 252 of the capture probe 250 bind to the pathogen to be detected by the system 200 according to embodiments of the invention. The pathogen, for example virus particles, in a liquid or gas (air) medium which is passed over the crystal microbalance, bind to the probes and are captured through the binding region, which is designed to be specific to the target, causing a change in mass on the microbalance.

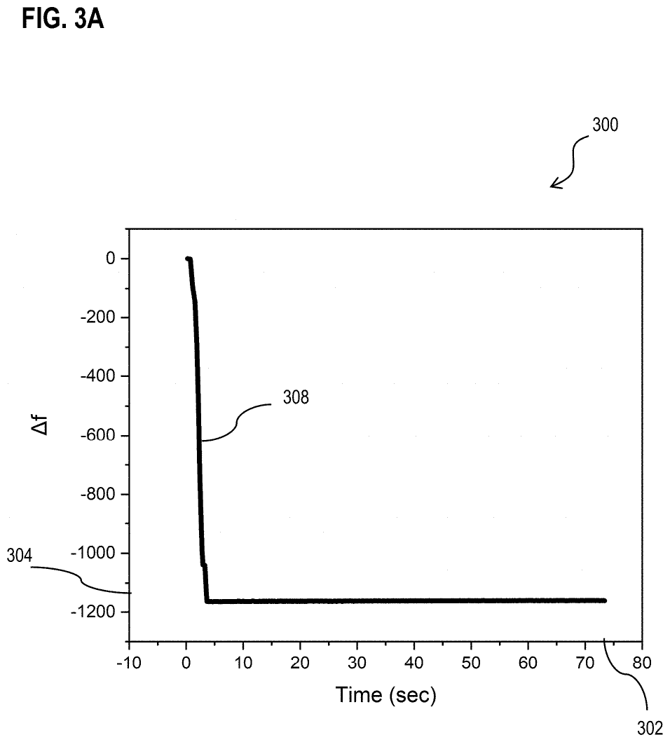

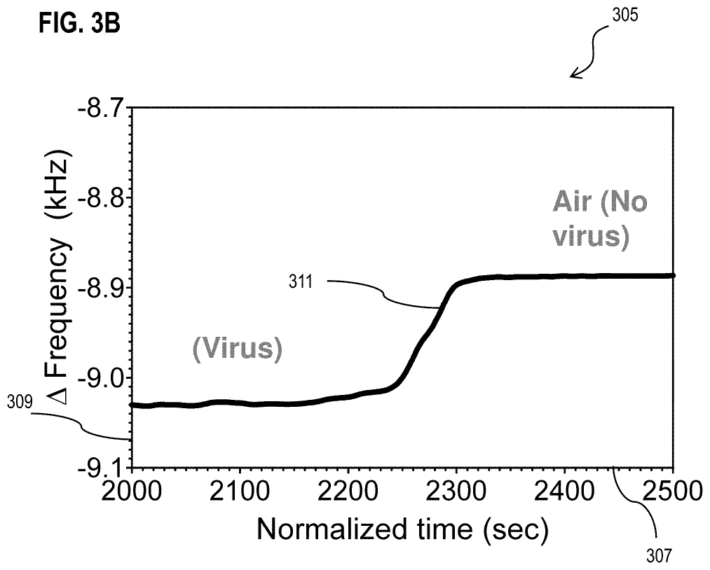

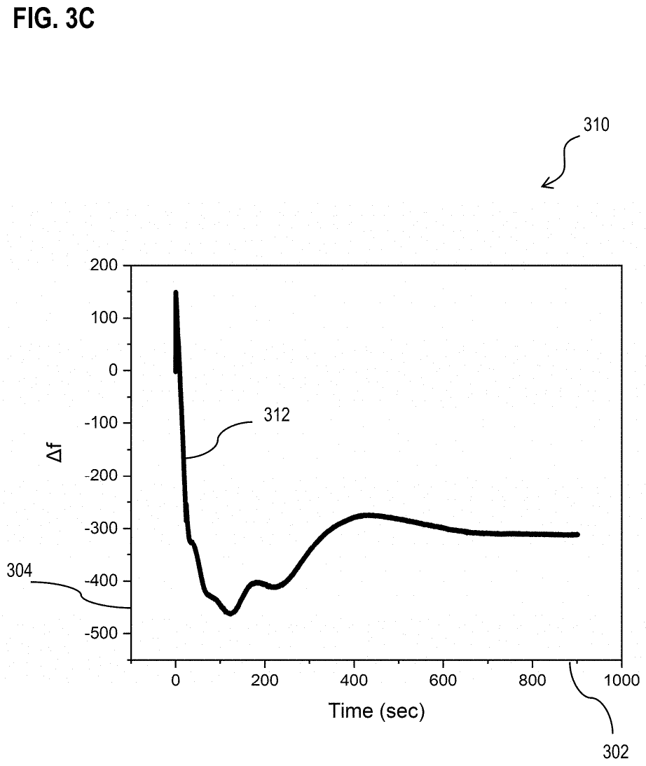

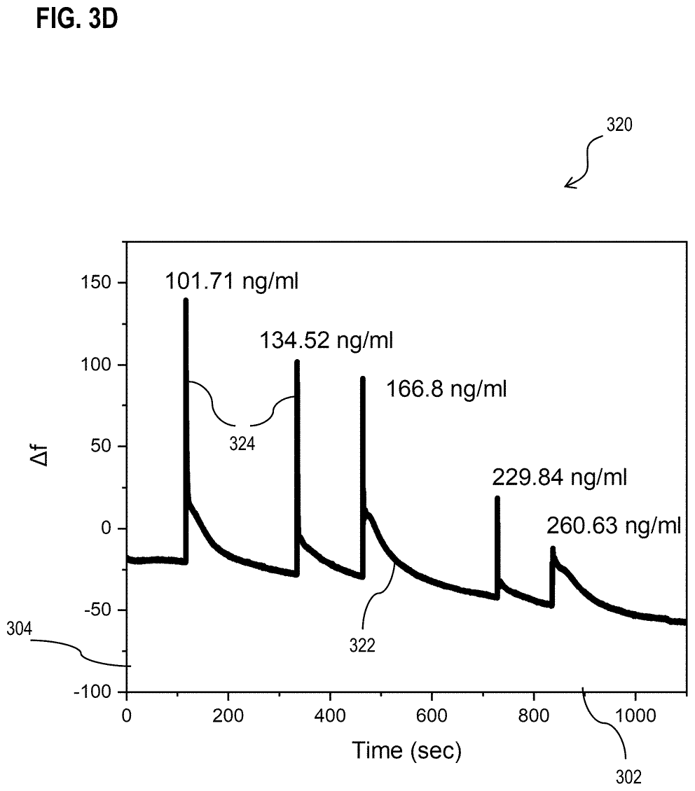

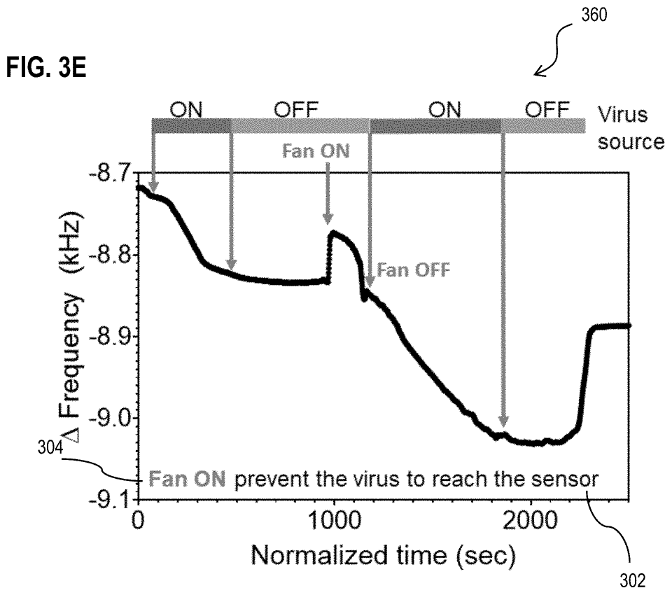

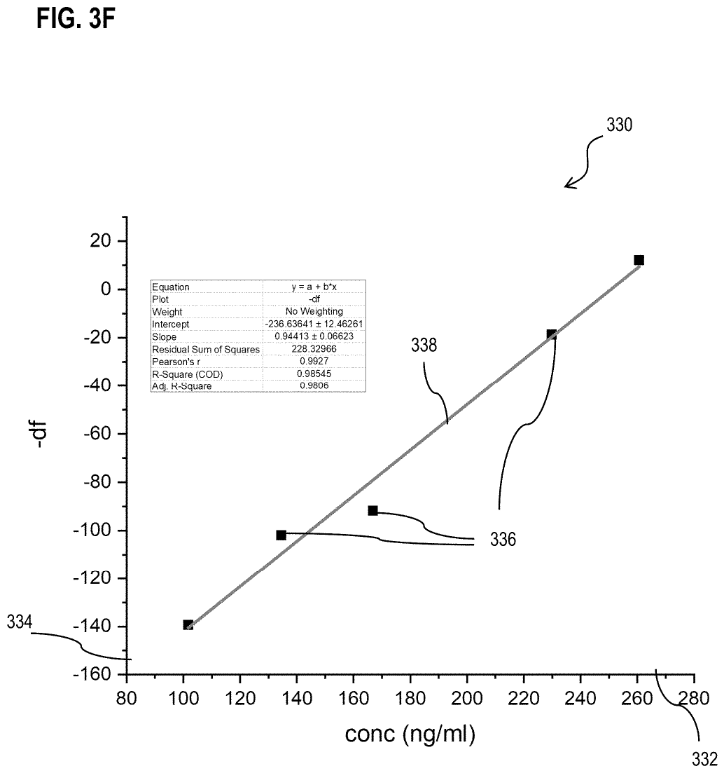

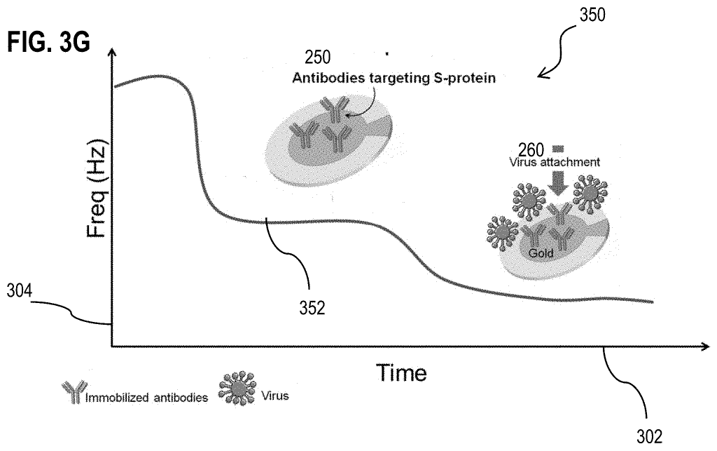

Calibration of the system 200 is now discussed, specifically calibrating the resonant frequency of the QCM 201 to added mass on the QCM 201. FIGS. 3A through 3G are graphs that illustrate an example of data obtained during a calibration of the system 200 of FIG. 2A, according to an embodiment. The graph 300 of FIG. 3A indicates how the resonant frequency of the QCM 201 shifts based on adding water to the QCM 201. The horizontal axis 302 is time in units of seconds (secs). The vertical axis 304 is resonant frequency shift in units of Hertz (Hz). The curve 308 indicates that after adding water to the QCM 201, the resonant frequency of the QCM 201 falls about −1100 Hz after which the resonant frequency remains approximately fixed. In an example embodiment, FIG. 3A depicts how the resonant frequency shifts based on adding water to the QCM, where the QCM is positioned in a liquid collector.

The graph 305 of FIG. 3B indicates how the resonant frequency of the QCM 201 shifts based on adding air (with no virus particles) to the QCM 201. The horizontal axis 307 is time in units of seconds. The vertical axis 309 is resonant frequency shift in units of kHz. The curve 311 indicates that after directing air (with no virus particles) on the QCM 201, the resonant frequency of the QCM 201 increases about 100 Hz (0.1 kHZ) after which the resonant frequency remains approximately fixed. In an example embodiment, FIG. 3B depicts how the resonant frequency shifts based on adding air (no virus particles) to the QCM, where the QCM is configured to perform dry air detection of virus particles in air directed at the QCM.

The QCM 201 is configured to indicate a change in the resonant frequency in the absence of a target biochemical that is substantially constant and greater than zero indicating the successful loading of the sensor with the capture probes 250 (e.g., SARS-CoV-2 S-antibodies), base state). In another embodiment, the QCM 201 is configured to avoid any non-specific binding by the surface, such as with a 3% Bovine Serum Albumin (BSA) blocking solution followed by rinsing. This reduces the likelihood of falsely causing a triggered state by binding of non-specific material to the sensor. FIG. 3C indicates a graph 310 with a curve 312 that indicates the shift in the resonant frequency upon adding 3% BSA to the QCM 201. In an embodiment, FIG. 3C represents the response of the sensor (e.g. QCM 201) after adding the blocking agent. In an embodiment, this step is performed regardless of whether the QCM 201 is in a liquid collector or is configured to perform dry air detection. In one embodiment, the use of the blocking agent (e.g., bovine serum albumin (BSA)) helps in avoiding the non-specific binding to the electrode surface. FIG. 3C shows that a change in the QCM frequency is observed after the addition of BSA. The QCM frequency reaches a constant value which does not change even after the addition of more concentration of BSA.

As an example of calibrating the system 200, different known masses of pathogen particles 260 are directed at the QCM 201 and the resulting resonant frequency of the crystal 202 is measured for each known mass of particles 260 to be detected. FIG. 3D shows a graph 320 which depicts this calibration data. The curve 322 indicates the measured resonant frequency shift for each known mass of pathogen particles 260 directed at the QCM 201. The peaks 324 on the curve 322 indicate the shift in the resonant frequency for each known mass of pathogen particles 260 labeled over each peak 324.

In some embodiments, calibration data similar to FIG. 3D is generated for the QCM 201 that is configured to perform dry air detection. In this embodiment, air with different known masses or concentration of pathogen particles 260 is directed at QCM 201 at different time periods. FIG. 3E depicts a graph that shows such calibration data. One or more pathogen particles 260 are directed at the QCM 201 using a means to direct a medium (e.g. air, liquid) containing the pathogen particles 260 at the QCM 201. In one embodiment, the means is a fan or other such device. The resonant frequency shift can be monitored after such means are activated to direct the pathogen particles 260 at the QCM 201. FIG. 3E is a graph that illustrates an example of a change in the resonant frequency of the QCM 201 of FIG. 2A based on directing a medium including the pathogen particles 260 at the QCM, according to an embodiment. As shown in the graph 360, the shift in the resonant frequency decreases after the fan is activated, since this accelerates the number of pathogen particles 260 that bind to the capture probes 250 in a given unit of time and thus increase the added mass on the QCM 201. Additionally, the frequency shift varies over time as the fan is activated, since air with different known masses or concentrations of pathogen particles 260 are directed at the QCM 201. Hence, during these time periods when the fan is activated, the shift in the resonant frequency increases in magnitude. As also shown in the graph 360, when the fan is deactivated the number of pathogen particles 260 that bind to the capture probes 250 decrease in a given unit of time and hence the amount of added mass to the QCM 201 decreases. Hence, during these time periods of the graph 360, the shift in the resonant frequency reduces in magnitude.

The data obtained from the graphs of FIGS. 3D and/or 3E includes data points 336 that include a known mass of pathogen particles 260 and a resulting resonant frequency shift for each known mass. These data points 336 are then arranged on the graph 330 of FIG. 3F and a best fit curve 338 (e.g. least square) is provided through these data points 336. The horizontal axis 332 is mass of the pathogen particles 260 in units of ng (dry air detection) or liquid concentration (QCM in a liquid collector) of pathogen particles in units of ng/ml. The vertical axis 334 is resonant frequency shift in units of Hertz. In an embodiment, this curve 338 is then stored in a memory of the controller 230 and is used to estimate a mass of pathogen particles 260 bound to the QCM 201 based on a measured resonant frequency shift. In an example embodiment, the controller 230 receives the data from the frequency detection unit 220 that indicates the shift in the resonant frequency. Based on these data, the controller 230 uses the curve 338 to determine the mass of pathogen particles 260 added to the QCM 201, i.e. the value along the horizontal axis 332 that corresponds to the resonant frequency shift along the vertical axis 334. In some embodiments, for systems where the QCM is used for dry air detection, the standard curve 338 can be used that was obtained for liquid collection and back calculated to a concentration of pathogen particles 260 in air but using a correction value (e.g. constant).

In an embodiment, the graph 320 of FIG. 3D depicts the shift in the crystal's resonance frequency (Δf) as a function of time with continuous change of the target concentration. In another embodiment, FIG. 3F depicts the standard curve of the sensor correlating the shift in frequency with changing viral load (e.g. changing S-protein concentration, which will be used to estimate the number of SARS-CoV-2 virus particles).

In an embodiment, the system discussed herein for detecting the airborne virus can be scaled up into a system for high-density clustered population. To this end, the inventors of the present invention utilized a Quartz crystal microbalance (eQCM) and functionalized it with SARS-CoV-2 S-protein specific antibodies. In an embodiment, a bioconjugation technique has been used to couple the antibodies to the electrode 204 (e.g. gold surface layer over the crystal or the capture membrane 720). Results were obtained which indicate that the change in resonance frequency in the absence of the target is constant and greater than zero indicating the successful loading of the QCM with the SARS-CoV-2 S-antibodies.

To avoid any non-specific binding to the electrode surface or capture membrane 720, 3% Bovine Serum Albumin (BSA) was used to block surface. FIG. 3C shows that the QCM reaches a stable point after 500 secs of the addition of BSA, and no further change was observed thus confirming saturation was reached. After blocking of the QCM surface, the sensor showed no obvious response to the addition of BSA, which further confirmed successful blocking of non-specific binding. To mimic the environment of airborne SARS-CoV-2 sampler system in a liquid chamber, a liquid cell was utilized where solutions of S-protein of different concentrations were used, and the change in the QCM resonance frequency recorded in each case. FIG. 3D depicts the response of the system with the continuous change in the S-protein concentration. This experimental protocol mimics the real-world scenario; if airborne SARS-CoV-2 is present in the air, the concentration of the virus will change in a continuous fashion. The sensor, in this case, will be used for the continuous monitoring of the virus without an intermediate washing step. The sensor was found to be responsive to different concentrations of the antigen with no obvious change as a response to the addition of an equal amount of water to the chamber (FIG. 3D). The calibration curve of the antigen concentration versus change in crystal resonance frequency is shown in FIG. 3F.

The calibration of the system 200 further includes accounting for the mass of the capture probes 250 added to the QCM 201, analogous to taring the QCM. FIG. 3G is a graph 350 that includes a curve 352 that indicates a shift in the resonant frequency of the QCM 201 based on conjugating the capture probes 250 to the electrode 204 and then a further shift in the resonant frequency based on binding pathogen particles 260 to the capture probes 250. In this example embodiment, the first shift in the resonant frequency due to the conjugation of the capture probes 250 to the QCM 201 is used as a baseline for assessing a change in the mass on the QCM 201 due to the added pathogen particles 260 that bind to the capture probes 250. In this example embodiment, the resonant frequency shift that is detected by the frequency detection unit 220 and controller 230 is measured from the resonant frequency baseline value where the capture probes 250 are conjugated to the QCM 201.

FIG. 4 is a flow diagram that illustrates an example method 400 to detect a pathogen load in a medium with the system 200 of FIG. 2A, according to an embodiment. Although steps are depicted in FIG. 4, in other embodiments, one or more steps, or portions thereof, are performed in a different order, or overlapping in time, in series or in parallel, or are omitted, or one or more additional steps are added, or the method is changed in some combination of ways.

In step 401, the system 200 is calibrated. In this step, the resonant frequency of the QCM 201 is calibrated to mass added to the QCM 201. As previously discussed with respect to FIGS. 3D through 3F, in step 401 different known masses of pathogen particles 260 are added to the QCM 201 (via binding to the capture probes 250) and the resulting resonant frequency shift is recorded for each known mass. A best linear fit curve is then generated based on this data which relates the mass change on the QCM 201 to the resonant frequency shift. In the embodiment of FIG. 3F, the curve 338 is generated which is stored in the memory of the controller 230.

In one embodiment, in step 402 the capture probes 250 are conjugated to a sensor chip that includes the QCM 201. In an example embodiment, for purposes of this description, “sensor chip” means a component of a system that is removable or replaceable and includes the QCM 201. In some embodiments, the sensor chip can be removed, washed and replaced in the system 200. In other embodiments, the sensor chip is disposable and can be replaced with a new sensor chip, such as when the amount of added mass (e.g. virus particles 260) added to the QCM 201 exceed a threshold mass such that adding further mass would not generate a measurable or detectable resonant frequency shift.

In an embodiment, the sensor chip is reusable. In one embodiment, a surface of the sensor chip (e.g. electrode 204) is functionalized with ssDNA/aptamers/peptides/antibody and once the experiment is done the sensor chip is washed with regeneration solution. For example, in the case of protein we can use one of the following: Low pH (10 mM glycine-HCl at pH 1.5 to 3, available as ready-to-use solutions from GE) is often appropriate for regenerating protein surfaces. Other conditions which have proved useful include: high pH (1 to 100 mM NaOH); high ionic strength (e.g. up to 5 M NaCl or 4 M MgCl2); low concentrations of SDS (up to 0.5%); and ethylene glycol at concentrations up to 100%.

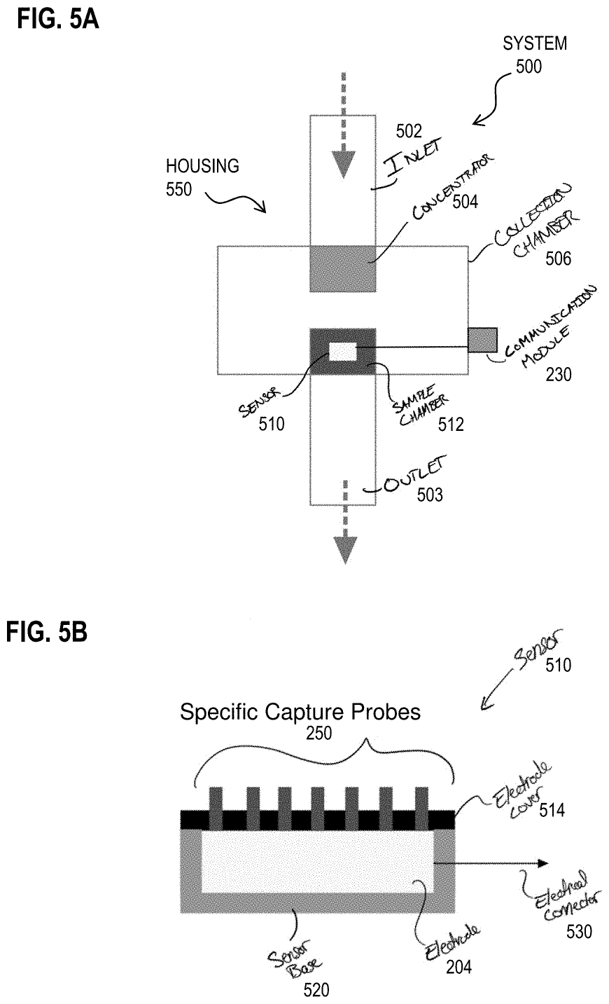

In step 404, the sensor chip is installed in a housing and a medium is directed at the sensor chip in the housing. In an embodiment, the medium is air that includes the pathogen particles 260. In an embodiment, the sensor chip includes a removable or replaceable QCM 201 that is installed in a housing and is used to detect the load of virus particles 260 or other pathogens in the medium that is directed at the QCM 201. FIGS. 5A and 5B are images that illustrate an example of a viral detection system 500 including a housing 550 to direct a medium at the QCM 201 of the system 500, according to an embodiment. As shown in FIG. 5A the housing 550 includes an inlet 502 through which a medium (e.g. air) is introduced. The sensor chip 510 is also depicted in FIG. 5A that includes the QCM 201 and is positioned within a collection chamber 506 of the housing 550. A concentrator 504 is also positioned adjacent the inlet 502 and is used to direct the medium at the sensor chip 510. As shown in FIG. 5B, the capture probes 250 are conjugated to an electrode cover 514 of the electrode 204. An electrical connector 530 is also depicted which connects the electrode 204 to the controller 230. After the medium including the pathogen particles 260 reaches the sensor chip 510 and the pathogen particles 260 bind to the capture probes 250 on the QCM 201, the medium (without the pathogen particles to be detected 260) exits the housing 550 through an outlet 503. This advantageously ensures that the medium exiting the housing 550 does not include the pathogen 260.

In step 406, a determination is made whether a total mass of pathogen particles 260 that have bound to the capture probes 250 exceed a total mass capacity for the sensor chip 510. Once the amount of added mass of pathogen particles 260 to the QCM 201 reaches the total mass capacity, further resonant frequency shift cannot be measured and thus the sensor chip 510 cannot detect a further added mass. In an example embodiment, in step 406 the total resonant frequency shift is monitored since a first added mass of virus particles 260 was added to the QCM 201. Once this total resonant frequency shift reaches a threshold shift, the sensor chip 510 cannot measure any further resonant frequency shift. In an example embodiment, upon determining in step 406 that the total mass of pathogen particles 260 exceeds the total mass capacity, the sensor chip 510 is removed and replaced with a new sensor chip 510. In other embodiments, in step 406 once the total mass of pathogen particles 260 exceeds the total mass capacity, the pathogen particles 260 that have bound to the capture probes 250 are removed so that the same sensor chip 510 can be used once these pathogen particles 260 are removed.

In step 408, for each measurement time the resonant frequency of the QCM 201 of the sensor chip is measured. In an embodiment, in step 408 the resonant frequency is measured by the frequency detection unit 220 and a signal is transmitted to the controller 230 that indicates the measured resonant frequency. In one embodiment, in step 408 the controller 230 determines a shift in the resonant frequency based on the measured resonant frequency received from the frequency detection unit 220 and a previous measured resonant frequency at a measurement time prior to the current measurement time. In an embodiment, in step 408 the controller 230 further determines a mass change on the QCM 201 based on the shift in the resonant frequency. In an example embodiment, in step 408 the controller 230 uses a best fit linear curve between the added mass and the resonant frequency shift to determine the added mass to the QCM 201 attributable to the resonant frequency shift. In an example embodiment, the controller 230 uses the curve 338 of FIG. 3F in determining the added mass. In other embodiments, the controller 230 uses equation 2 to determine the change in mass on the QCM 201 based on the measured resonant frequency shift.

In step 410, the controller 230 then relates the mass change from step 408 with a viral load in the medium (e.g. air) directed at the QCM 201. In an embodiment, in step 410 the controller 230 uses a molecular weight for the pathogen particle 260 to relate the mass change with the viral load in the medium. In this example embodiment, the viral load is expressed as a total number (N) of pathogen particles 260 that bound to the specific capture probes 250 during the measurement time. In an example embodiment, in step 410 the controller 230 uses the following equation:

$$N = \frac{Δ m}{ρ V} \qquad ( 3 )$$

Where Δm is the change in mass determined in step 408, ρ is the volume density of a pathogen particle 260 and V is the volume of a pathogen particle 260. In another embodiment, in step 410 the controller 230 generates a curve similar to the curve 352 of FIG. 3G that relates the resonant frequency shift over time. In this embodiment, in step 410 the controller 230 determines the viral load in the medium based on a value of a slope of the curve at any given time. This slope indicates a time rate of change of the resonant frequency shift which is proportional to a time rate of change of the added mass of pathogen particles 260 to the QCM 201.

In step 412, the controller 230 determines whether the viral load from step 410 is greater than a high load threshold. In an embodiment, the high load threshold from step 412 is stored in a memory of the controller 230 and indicates a condition of an area from which the medium was gathered (e.g. that the area is contaminated with the virus, that individuals in the area need to be quarantined, etc.). In an example embodiment, a high load threshold of the viral load corresponds to a shift in the resonant frequency of more than 100 Hz. If the determination in step 412 is in the affirmative, the method 400 moves to block 414. If the determination in step 412 is in the negative, the method 400 moves back to block 406 since this indicates that there is not an excessive viral load in the medium and thus there is no need to issue a notification of an excessive viral load.

In step 414, the controller 230 notifies a user of the system of an excessive viral load in the medium based on the determination in step 412. In one embodiment, in step 414 the controller 230 outputs a visual output on a display (e.g. display 914 of FIG. 9) or outputs an audible alert with an audible device (e.g. speaker).

### 3. EXAMPLE EMBODIMENTS

Some example embodiments are now discussed, including some example embodiments of various housings that can be used to deliver medium (e.g. air, liquid) including pathogen particles at the QCM. In an embodiment, the system takes a sample of the air and then pass it through a filter to remove dust and debris. Next, the presence of the virus in the sampled air will be detected through an electrical sensing chip (that includes the QCM). The electrical chip transduces the binding event of the pathogen particle 260 to the antibody or other specific probe 250 into a detectable electrical signal. This can be observed as a shift in the main resonant frequency of the chip (e.g. resonant frequency of the quartz crystal 202) and found to correlate with the virus concentration. Finally, the air will pass through multiple filters to remove the pathogen particles 260 (e.g. which did not bind to the capture probes 250) and bacteria to eject fresh air. In an example embodiment, the system shows an instantaneous response and can detect COVID-19 within a short time (e.g., about 4.5 minutes) past the release of the SARS-CoV-2 virus in a room by an infected subject. The sensor chip is configured for continuous monitoring of viral presence in an area without an intermediate washing step. The sensor chip is further configured to be responsive to different concentrations of the antigen (e.g., viral presence) with no or low obvious change as a response to the addition of an equal amount of aerosol to the chamber.

The fundamental advantages offered by the system include one or more of: detection is relying on capturing the whole virus and the electrical signal is based on a proprietary sensor surface for recognition of the biological binding events; no amplification is necessary; no heating step or time delay; directly detects the pathogen from the air and no condensation or processing step is necessary; essentially a real-time detection of the virus; modularity and multiplexing with emerging viruses and bacterial pathogens; much simpler to adopt and integrate for emerging pathogens than nucleic acid-based (LAMP) techniques; air goes in and gets sensed (pathogens get detected) and travels through a set of HEPA filters for purification; and portable and can be wirelessly connected and the data can be transmitted in real-time for analysis.

The system is configured to collect airborne viral molecules to measure viral presence. In an embodiment, the system includes a housing, at least one filter, at least one fan, and a sensor (e.g. sensor chip including the QCM). The housing is configured to house components, such as at least one filter, at least one fan, and the sensor, and to direct airflow there through. The housing can include an inlet and an outlet, for example, as a plurality of apertures that allow airflow from an environment to the components.

For example, the system can take a sample of air and remove dust (e.g., filter dust particles through a filter to remove dust and debris). The presence of viral components in the sampled air is detected through an electrical sensing chip (e.g. QCM), which includes a capture probe 250 (e.g. antibodies). Tested was performed a simulated system and virus spread as aerosols (nebulizer virus concentration: 6.07E+06 IFUs/mL) with a nebulizer flow rate of 2.00E-03 mL/min. Within 10 seconds, the sensor (time of response) the amount of the virus ejected in the box within the sensor's TOR was 2.02E+03 IFUs with a calculated LOD of 79.69 viral copies (IFUs)/L.

In addition, several signal processing algorithms such as empirical mode decomposition (EMD), Wavelet Transform (WT), and others would be investigated to decompose the signal. This would allow revealing the subtle change in the electrical signal associated with a low virus concentration. Further, the embodiments of the present invention include developing a resonator-based sensor array for the detection of multiple pathogens at once.

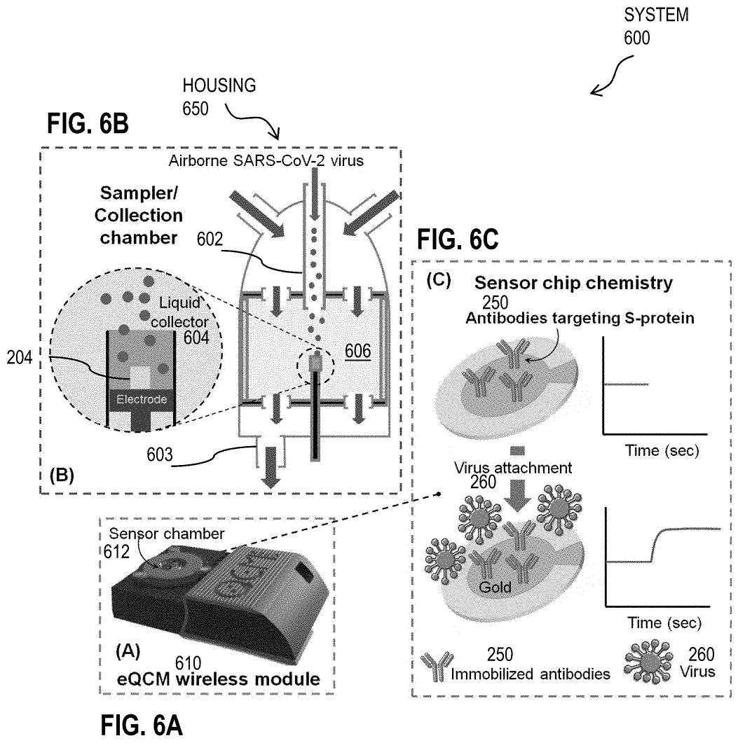

FIGS. 6A through 6C are images that illustrates example of a viral detection system 600 including a housing 650 to direct a medium at the sensor chip of the system 600 that is mounted in a sensor chamber 612 of a QCM wireless module 610. In an embodiment, the QCM of the QCM wireless module 610 includes the electrode 204 positioned in a liquid collector 604 which is mounted in a collection chamber 606. The pathogen particles 260 are collected in the liquid collector 604 and subsequently bind to the capture probes 250 that are conjugated to the electrode 204. Thus, the embodiment of FIGS. 6A through 6C (liquid collection at the electrode 204) differs from the embodiment of FIGS. 5A through 5B (air collection at the electrode 204). In an embodiment, the wireless module 610 can either feature an electrical connector similar to the connector 530 of FIG. 5B to connect the sensor chip to the controller 230 or the wireless module 610 itself is a housing that encloses the controller 230. The housing 650 features an outlet 603 that directs the medium (without the pathogen particles 260) to the area outside the housing 650.

In a preferred embodiment, FIG. 6A depicts an eQCM wireless module 610 where the developed crystal chip sensor (e.g. QCM) is integrated and used for detecting airborne SARS-CoV-2. The module 610 is connected wirelessly with online Cloud for further analysis. In another preferred embodiment, FIG. 6B depicts electrostatic precipitation used to collect the airborne virus into a liquid collector 604 which will be integrated with the QCM wireless module 610. Suitable liquids for use in the liquid collector 804 include water, aqueous saline solution, or the like, optionally also containing anti-fungal or anti-bacterial compounds when the target pathogen is a virus. In another embodiment, FIG. 6C depicts the operating principle of the QCM-based airborne virus detector. In the absence of the airborne pathogen particles 260 the sensor shows a stable signal with no shift in the resonance frequency. In the presence of the pathogen particles 260, a shift of the crystal resonance frequency is observed which is correlated with the virus concentration. In the embodiment, the system 600 consists of three main subunits, as shown in FIGS. 6A through 6C, which are: 1) the airborne collection system, 2) the filtering unit, 3) the QCM based sensor. In an example embodiment, the inventors of the present invention developed a sensor chip for highly specific S-protein-based recognition of SARS-CoV-2 virus (antigen test). In another example embodiment, another sensor chip is provided that contains capture probes specific for influenza A H1N1 hemagglutinin (HA), for example an H1N1 HA-targeted monoclonal antibody. The QCM sensor therefore can provide a detectable signal in terms of change in frequency for SARS-CoV-2, distinguishable from the seasonal influenza A virus.

As shown in FIGS. 5A-5B and FIGS. 6A-6C, one embodiment of the device is an antigen-based sensing platform configured to measure and communicate the presence of a virus in an area, such as an indoor or enclosed space. The device is configured to collect ambient air (or an air sample) from the area, measure the air sample to determine presence of the virus (or any microbe, pathogen, etc.), and communicate results of the measurement to a user or external device (via a communication module). Thus, the device can increase the likelihood of detecting the presence of a pathogen such that users and authorities have data to take actions.

One embodiment of the device includes a housing, an airborne collecting system, a filtering unit, a sensor, and a communication module. Each component of the device is configured to increase the measurement of viral presence in the area. In one embodiment, the housing has an inlet and an outlet configured to manage flow of an air sample, such as having pathogen particles, toward the sensor. Accordingly, the filtering unit is configured to provide the sensor with an air sample with a sufficiently low concentration of particles of which the sensor is not configured to measure, thus reducing interference (or noise) from the particles that could reduce the effectiveness of the sensor measuring for the presence of a virus. For example, in one embodiment the filtering unit includes multi-layer filters configured to remove large particles and debris present in the environment, while in other embodiments, the filtering unit is a single layer filter. Still referring to FIGS. 5A-5B and FIGS. 6A-6C, the sensor of one embodiment is an eQCM sensor for virus capturing.

Still referring to FIGS. 6A through 6C, the sensor is configured to measure and provide a signal of presence of a virus in an air sample. In the current embodiment, the sensor is configured to measure specific viruses (or microbes or pathogens, etc.) by detecting biochemical signatures of a specific virus in an air sample. For example, the biochemical signatures can be DNA-based, RNA-based, protein-based, or the like. In the current embodiment, the sensor is configured to measure for biochemical signatures of S-proteins for recognition of SARS-CoV-2 virus (i.e. antigen test). In another embodiment, the sensor chip can be configured to measure and detect presence of other viruses, such as influenza A H1N1 hemagglutinin (HA) (e.g., using targeted monoclonal antibodies).

Specifically referring to FIG. 6C, in one embodiment the sensor is configured to measure and detect presence of a virus by detecting a change in a signal. For example, in a base state the sensor provides a signal having parameters, such as frequency or amplitude, indicating lack of a (or substantially low) viral presence in an air sample. In a triggered state, the sensor provides a signal having parameters indicating viral presence in an air sample. In the current embodiment, the device is in a base state providing a signal with stable form and without a shift in the signal's resonance frequency, thus indicating absence of a virus. In the presence of a virus, however, the device is in a triggered state providing a signal having a shift of its resonance frequency that is correlated to a concentration of a virus in the air sample.

Still referring to FIGS. 6A through 6C, in an embodiment the sensor of the current embodiment is based on a quartz crystal microbalance (QCM) mechanism configured to provide a detectable signal having a change in frequency indicating detection of a virus, such as SARS-CoV-2, seasonal influenza A virus, or the like. The QCM mechanism includes the electrode 204 and a plurality of capture probes 250 immobilized on the electrode 204 (FIGS. 2A-2B). In a preferred embodiment, the electrode 204 is a gold-based material. The capture probes 250 are chosen to so as to selectively bind to a targeted portion of a specific virus particle 260 (or microbe or pathogen). In a preferred embodiment, for example, the capture probes 250 are selected to bind to S-proteins of a SARS-CoV-2 virus. In other embodiments, the capture probes 250 are chosen to bind to other molecules or antigens of a virus or microbe 260, such as Hemagglutinin (HA) surface protein of the influenza virus or anti-E. coli antibody, anti-B. anthracis antibody, anti-Salmonella typhimurium antibody, etc. Other targeting ligands such as peptides can also be used.

As discussed above, the sensor has a base state and a triggered state that provide a signal indicating presence of a virus in an air sample. The plurality of capture probes 250 of the QCM 201 mechanism are fixed and electrically coupled to the electrode 204 as components of an electrical circuit. In an embodiment, the plurality of capture probes 250 affect resonance frequencies of the electrical circuit based on whether the capture probes 250 are attached or unattached to target biochemical (e.g. pathogen particle 260). For example, the electrical circuit can have a higher resonance frequency when a plurality of capture probes 250 are attached to a target biochemical (e.g. pathogen particle 260), such as S-proteins of a SARS-CoV-2 virus, compared to a resonance frequency when a plurality of capture probes 250 are unattached to a target biochemical. The QCM mechanism 201 provides a signal having a particular resonance frequency, as noted above. In the current embodiment, a signal having a higher resonance frequency corresponds to the triggered state, while a signal having a lower resonance frequency corresponds to the base state. In these embodiments, measuring the signal (e.g., the signal's frequency) provided by the QCM mechanism 201 indicates viral presence. Thus, the QCM mechanism 201 is configured to provide a signal indicating presence of a virus in an air sample.

According to the current embodiment, the system 600 is configured as a reusable viral detection device. For example, the sensor chip of the system 600 can be reset from a triggered state to a base state by washing the sensor chip with washing buffer. In another embodiment, the sensor chip is a one-time use, such that the sensor chip can is discarded (e.g., when reaching a chosen level of measurement of viral presence or reaching a triggered state). According to one embodiment, the washing buffer is a solution appropriate for regenerating protein surfaces. For example, the washing buffer may have low pH, such as 10 mM glycine-HCl at pH 1.5 to 3. In other embodiments, the washing buffer is configured to have high pH (e.g., approximately 1 to 100 mM NaOH), high ionic strength (e.g., approximately up to 5 M NaCl or 4 M MgCl2, low concentrations of SDS (approximately up to 0.5%), or ethylene glycol (e.g., approximately concentrations up to 100%).

Still referring to FIGS. 6A through 6C, another embodiment of the device is configured for increased robustness for providing a signal indicating presence of a virus in an air sample, compared to typical devices. For example, the sensor can be configured to compensate for interfering signals from the environment or external noise. In one embodiment configured for increased robustness, the sensor includes a plurality of QCM mechanisms wherein at least one of the QCM mechanisms is a reference (i.e. “dummy element”) mechanism. The device can be further configured to manipulate the signals from the plurality of QCM mechanisms to suppress interfering signals or external noise. For example, the device can be configured to computationally process the signals. In another embodiment, the signals from the plurality of QCM mechanisms are communicated to an external device to computationally process the signals compensate for interfering signals from the environment or external noise.

According to the current embodiment, the device includes the communication module 610 that is configured to transfer signals (of data) indicating viral presence of an air sample to an external device (e.g., controller 230, such as a mobile device, computer, or server). For example, in one embodiment the communication module is a wireless communication module 610 configured to operate according to typical wireless communication standards (e.g., Wi-fi, Bluetooth®, cellular, and the like).

As discussed above, the sensor is configured to measure the viral presence in an air sample from an area, such as areas having a high-density clustered population. The sensor includes the QCM further being functionalized with specific capture probes for detecting and measuring corresponding viruses, such as SARS-CoV-2 S-proteins, or RNA or DNA viruses like coronaviruses, MERS, or Ebola, and the like. The QCM preferably is functionalized with protein-specific antibodies using a bioconjugation technique to couple the capture probes to the gold electrode of the QCM crystal or to the optional capture membrane. The capture probes can include single-stranded DNA (or oligonucleotide) probes, aptamers, peptides, and/or antibodies. Preferred examples of techniques for conjugation of the capture probes to the sensor device/electrode/capture membrane include surface activation with moieties such as thiol functional groups followed by EDC/NHS chemistry, click chemistry, conjugation linkers such as DSSTP, biotin-streptavidin, or thiol substitution.

According to some embodiments, the sensor includes ssDNA as selective capture probes to specifically bind pathogens. In still another embodiment, the ssDNA is conjugated to the QCM gold electrode.

The sensor is configured to indicate a change in resonance frequency in the absence of a target biochemical that is substantially constant and greater than zero indicating the successful loading of the crystal with the SARS-CoV-2 S-antibodies (i.e. base state). In another embodiment, the sensor is configured to avoid any non-specific binding region by blocking non-specific sites, such as by exposing the surface of the electrode to a blocking solution as known in the art, such as a 3% Bovine Serum Albumin (BSA) solution to reduce the likelihood of falsely causing a triggered state.

Now referring to FIGS. 5A-5B and FIGS. 6A-6C, according to another embodiment, the device includes a liquid chamber 604 configured to capture viral samples in the air sample for sensing. The device includes the liquid cell having chosen concentration of S-protein in which the sensor (e.g. QCM including the electrode 204) is placed within the liquid chamber. The sensor, as described above is configured to provide a signal having a resonance frequency, for example a resonance frequency that correlates to the base state or the trigger state. Thus, the sensor is configured to measure resonance frequency of the QCM for various S-protein concentrations.

Still referring to FIGS. 5A and 5B and FIGS. 6A-6C, the housing can further include a concentrator 504 configured to manipulate the air sample to concentrate viruses (or antigens) within the air sample toward the sensor 510, 610. In one embodiment, the concentrator is an electrostatic precipitator configured to exert a force (such as an electrostatic or electrodynamic force) to guide pathogen particles 260 in the air sample toward the sensor. For example, FIG. 6B illustrates the housing 650 having electrostatic precipitator on an interior surface of the housing, such as a vertical interior surface. The electrostatic precipitator is electrically charged to exert an electrostatic force on charged particles (e.g., particles with electrical moments) within the housing 650. In one example, the electrostatic precipitator has a gradient configured to exert an electrostatic force that varies along a vertical axis of the housing 650 (e.g., such as varying from the inlet 602 toward the outlet 603 of the housing 650) to better guide the pathogen particles 260 toward the sensor 610. In another embodiment the concentrator 504 is an air management device, such as a fan, baffles, flow guides, or the like, configured to guide pathogen particles 260 in the air sample toward the sensor 510.

Now referring to FIG. 3D, the sensor can have a response to a continuous change of the S-protein concentration in the device. FIG. 3D shows a generally linear relationship (according to several parameters like slope, intercepts, and the like) between the concentration of S-protein present in the air sample and the sensor signal. In other embodiments, the relationship may have various parameters that correlate the viral presence (e.g., concentration) in the air sample and the sensor signal. Such parameters are generally determined by calibration and testing. In another example, the sensor signal over time correlates to a continuous change of S-protein concentration, which mimics a real-world scenario in which airborne SARS-CoV-2 or other virus is present in the environment. In still another example, the sensor signal over time correlates to a cumulative effect of S-protein concentration in an environment. Thus, the sensor is configured for continuously monitoring for viral presence in an area without an intermediate washing step. The sensor is further configured to be responsive to different concentrations of the antigen (e.g., viral presence) with no or low obvious change as a response to the addition of an equal amount of water to the chamber (FIG. 3D). The calibration curve of the antigen concentration versus change in crystal resonance frequency is shown in FIG. 3F.

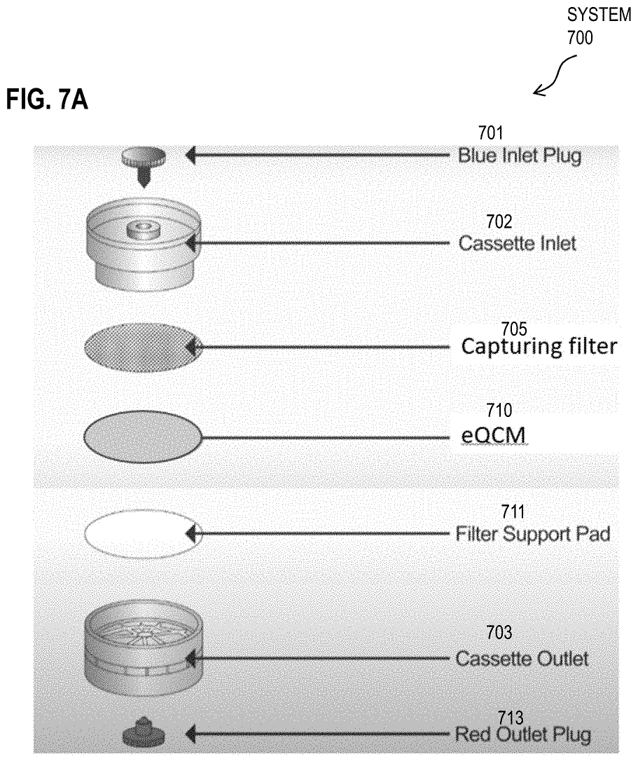

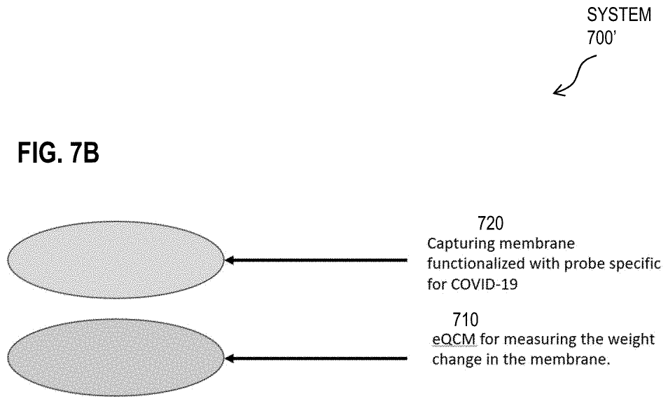

Another embodiment of a housing that is used to direct medium at the QCM is now discussed. FIGS. 7A and 7B are images that illustrates example of a viral detection system 700 including a housing to direct a medium at the QCM of the system 700, according to an embodiment. In an embodiment, the system 700 includes an inlet plug 701 and an outlet plug 713 which are removed during operation of the system 700 (so that the medium can flow through the inlet 702 to the QCM and subsequently the medium without the pathogen particles can exit the system at the outlet 703). Additionally, the system 700 includes a capturing filter 705 that is configured to remove debris and dust so that only the pathogen particles 260 pass through the filter 705. The system 700 also includes the QCM 710 to which the capture probes 250 are conjugated to bind to the pathogen particles 260. The system 700 further includes a filter support pad 711 that is configured to remove any pathogen particles 260 (which did not bind to the capture probes 250 of the QCM 710) before the medium exits the system 700 at the outlet 703. In another embodiment, a system 700′ of FIG. 7B is similar to the system 700 of FIG. 7A with the exception that the system 700′ includes a capturing membrane 720 on which the capture probes 250 are conjugated. In this embodiment, the system 700′ features the QCM 710 that is configured to measure a change in mass in the membrane 720 (due to pathogen particles 260 that bind to capture probes 250 conjugated to the membrane 720).

An embodiment of the invention includes a miniaturized capturing system, illustrated in FIGS. 7A-7B. The system 700 includes a housing, at least one filter 705, 711, and a sensor (e.g. eQCM 710). In one embodiment, the housing is configured to house and protect the system 700. In another embodiment the housing is configured to control or direct airflow from an environment to the sensor (e.g. eQCM 710). The housing includes an inlet 702 (such as a cassette inlet) and an outlet 703 (such as an outlet). Furthermore, the housing can include at least one plug 701, 713, for example, to control airflow and to protect other components, such the at least one filter 705, 711 and the sensor 710. In one embodiment, the sensor 710 is an electrochemical sensor, such as described above. In other embodiments, a system is provided to monitor and assess risk of a viral presence (e.g., such as COVID-19). The system receives data from an airborne sensor (such as the QCM discussed herein) and from external sources (e.g., from EMR, health applications, wearable sensors, symptoms input, etc.). The system is further configured to analyze the data, such as on a computer or a cloud system. In one embodiment, the system includes a machine learning algorithm using the data for assessing the risk of viral presence.

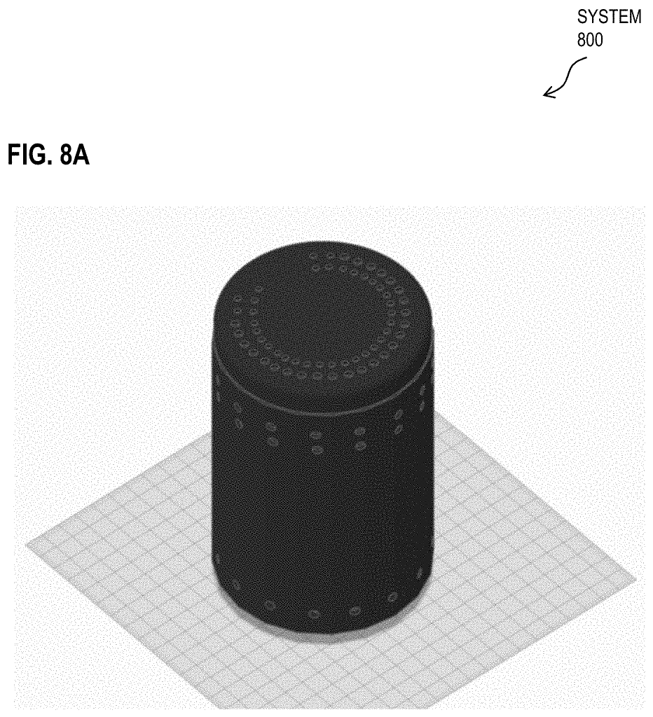

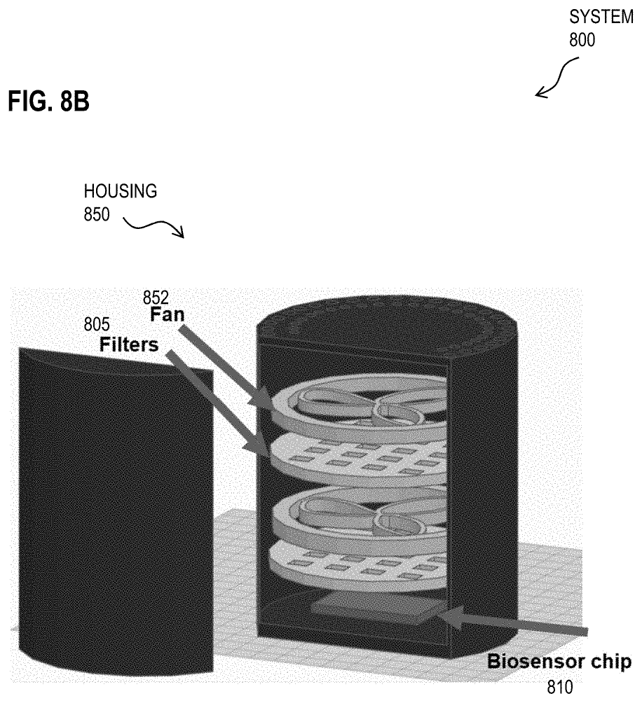

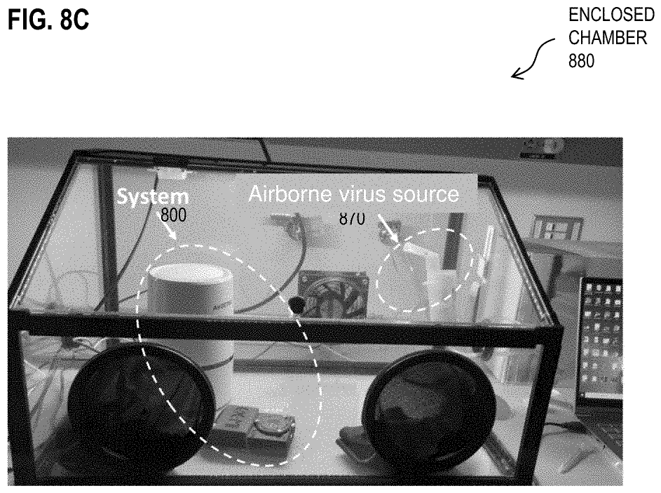

FIGS. 8A through 8C are images that illustrates example of a viral detection system 800 including a housing 850 to direct a medium at the QCM of the system 800, according to an embodiment. As shown in FIG. 8B, in an embodiment, the housing 850 features an inlet in a top of the housing 850 (e.g. holes in the top of the housing 850) and an inlet adjacent a base of the housing 850 (e.g. holes adjacent a base of the housing 850). The housing 850 also includes one or more fans 852 and filters 805. In an example embodiment, the fans 852 are configured to direct the medium (e.g., air) that enters the housing 850 through the inlet toward the QCM (e.g. biosensor chip 810). Additionally, in an example embodiment the filter 805 are similar to the filter 705 of the system 700 and thus are used to remove dust or debris (so that only the pathogen particles 260 pass through the filter 805 to the QCM (e.g. biosensor chip 810). In another embodiment, the filter 805 is similar to the filter 711 of the system 700 and is used to remove any pathogen particles 260 (e.g. which did not bind to the capture probes 250) before the medium exits the housing 850 at the outlet.

FIG. 8C depicts an arrangement where the housing 800 of FIGS. 8A and 8B is used to calibrate the QCM, such as in step 401 of the method 400. In this embodiment, the system 800 is positioned in an enclosed chamber 880. Additionally, a source of airborne virus 870 is positioned within the enclosed chamber 880. The source of airborne virus 870 is configured to direct a known mass of virus particles 260 at the system 800 during a certain time period. This is then used to calibrate the system 800 by correlating the known mass of virus particles 260 to the measured resonant frequency during the same time period. Based on these measurements, the calibration data similar to what was depicted in FIGS. 3D through 3F is generated for the system 800. The controller 230 of the system 800 is then used to store the best fit curve (e.g. curve 338 of FIG. 3F) in the memory of the controller 230. The inventors recognized that an advantage of using the enclosed chamber 880 is to ensure that only the specific virus particles 260 to be detected affect the mass change on the QCM and to prevent contamination of the area where the enclosed chamber 880 is located.

For example, the system 800 can take a sample of air and remove dust (e.g., filter dust particles through a filter to remove dust and debris). The presence of viral components in the sampled air is detected through an electrochemical sensing chip 810, such as described above. In some embodiments, the sensor 810 includes at least one capture probe 250, while in other embodiments the sensor can include an impedance detection or capacitance detection. In still other embodiments, the system includes multiple filters 805 to substantially remove viruses and bacteria before discharging the medium from the housing 850.

### 4. HARDWARE OVERVIEW

FIG. 9 is a block diagram that illustrates a computer system 900 upon which an embodiment of the invention may be implemented. Computer system 900 includes a communication mechanism such as a bus 910 for passing information between other internal and external components of the computer system 900. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 900, or a portion thereof, constitutes a means for performing one or more steps of one or more methods described herein.

A sequence of binary digits constitutes digital data that is used to represent a number or code for a character. A bus 910 includes many parallel conductors of information so that information is transferred quickly among devices coupled to the bus 910. One or more processors 902 for processing information are coupled with the bus 910. A processor 902 performs a set of operations on information. The set of operations include bringing information in from the bus 910 and placing information on the bus 910. The set of operations also typically include comparing two or more units of information, shifting positions of units of information, and combining two or more units of information, such as by addition or multiplication. A sequence of operations to be executed by the processor 902 constitutes computer instructions.

Computer system 900 also includes a memory 904 coupled to bus 910. The memory 904, such as a random access memory (RAM) or other dynamic storage device, stores information including computer instructions. Dynamic memory allows information stored therein to be changed by the computer system 900. RAM allows a unit of information stored at a location called a memory address to be stored and retrieved independently of information at neighboring addresses. The memory 904 is also used by the processor 902 to store temporary values during execution of computer instructions. The computer system 900 also includes a read only memory (ROM) 906 or other static storage device coupled to the bus 910 for storing static information, including instructions, that is not changed by the computer system 900. Also coupled to bus 910 is a non-volatile (persistent) storage device 908, such as a magnetic disk or optical disk, for storing information, including instructions, that persists even when the computer system 900 is turned off or otherwise loses power.

Information, including instructions, is provided to the bus 910 for use by the processor from an external input device 912, such as a keyboard containing alphanumeric keys operated by a human user, or a sensor. A sensor detects conditions in its vicinity and transforms those detections into signals compatible with the signals used to represent information in computer system 900. Other external devices coupled to bus 910, used primarily for interacting with humans, include a display device 914, such as a cathode ray tube (CRT) or a liquid crystal display (LCD), for presenting images, and a pointing device 916, such as a mouse or a trackball or cursor direction keys, for controlling a position of a small cursor image presented on the display 914 and issuing commands associated with graphical elements presented on the display 914.

In the illustrated embodiment, special purpose hardware, such as an application specific integrated circuit (IC) 920, is coupled to bus 910. The special purpose hardware is configured to perform operations not performed by processor 902 quickly enough for special purposes. Examples of application specific ICs include graphics accelerator cards for generating images for display 914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 900 also includes one or more instances of a communications interface 970 coupled to bus 910. Communication interface 970 provides a two-way communication coupling to a variety of external devices that operate with their own processors, such as printers, scanners and external disks. In general the coupling is with a network link 978 that is connected to a local network 980 to which a variety of external devices with their own processors are connected. For example, communication interface 970 may be a parallel port or a serial port or a universal serial bus (USB) port on a personal computer. In some embodiments, communications interface 970 is an integrated services digital network (ISDN) card or a digital subscriber line (DSL) card or a telephone modem that provides an information communication connection to a corresponding type of telephone line. In some embodiments, a communication interface 970 is a cable modem that converts signals on bus 910 into signals for a communication connection over a coaxial cable or into optical signals for a communication connection over a fiber optic cable. As another example, communications interface 970 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 970 sends and receives electrical, acoustic or electromagnetic signals, including infrared and optical signals, that carry information streams, such as digital data.

The term computer-readable medium is used herein to refer to any medium that participates in providing information to processor 902, including instructions for execution. Such a medium may take many forms, including, but not limited to, non-volatile media, volatile media and transmission media. Non-volatile media include, for example, optical or magnetic disks, such as storage device 908. Volatile media include, for example, dynamic memory 904. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 902, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 902, except for carrier waves and other signals.

Logic encoded in one or more tangible media includes one or both of processor instructions on a computer-readable storage media and special purpose hardware, such as ASIC *920.

Network link 978 typically provides information communication through one or more networks to other devices that use or process the information. For example, network link 978 may provide a connection through local network 980 to a host computer 982 or to equipment 984 operated by an Internet Service Provider (ISP). ISP equipment 984 in turn provides data communication services through the public, world-wide packet-switching communication network of networks now commonly referred to as the Internet 990. A computer called a server 992 connected to the Internet provides a service in response to information received over the Internet. For example, server 992 provides information representing video data for presentation at display 914.

The invention is related to the use of computer system 900 for implementing the techniques described herein. According to one embodiment of the invention, those techniques are performed by computer system 900 in response to processor 902 executing one or more sequences of one or more instructions contained in memory 904. Such instructions, also called software and program code, may be read into memory 904 from another computer-readable medium such as storage device 908. Execution of the sequences of instructions contained in memory 904 causes processor 902 to perform the method steps described herein. In alternative embodiments, hardware, such as application specific integrated circuit 920, may be used in place of or in combination with software to implement the invention. Thus, embodiments of the invention are not limited to any specific combination of hardware and software.

The signals transmitted over network link 978 and other networks through communications interface 970, carry information to and from computer system 900. Computer system 900 can send and receive information, including program code, through the networks 980, 990 among others, through network link 978 and communications interface 970. In an example using the Internet 990, a server 992 transmits program code for a particular application, requested by a message sent from computer 900, through Internet 990, ISP equipment 984, local network 980 and communications interface 970. The received code may be executed by processor 902 as it is received, or may be stored in storage device 908 or other non-volatile storage for later execution, or both. In this manner, computer system 900 may obtain application program code in the form of a signal on a carrier wave.

Various forms of computer readable media may be involved in carrying one or more sequence of instructions or data or both to processor 902 for execution. For example, instructions and data may initially be carried on a magnetic disk of a remote computer such as host 982. The remote computer loads the instructions and data into its dynamic memory and sends the instructions and data over a telephone line using a modem. A modem local to the computer system 900 receives the instructions and data on a telephone line and uses an infra-red transmitter to convert the instructions and data to a signal on an infra-red a carrier wave serving as the network link 978. An infrared detector serving as communications interface 970 receives the instructions and data carried in the infrared signal and places information representing the instructions and data onto bus 910. Bus 910 carries the information to memory 904 from which processor 902 retrieves and executes the instructions using some of the data sent with the instructions. The instructions and data received in memory 904 may optionally be stored on storage device 908, either before or after execution by the processor 902.

FIG. 10 illustrates a chip set 1000 upon which an embodiment of the invention may be implemented. Chip set 1000 is programmed to perform one or more steps of a method described herein and includes, for instance, the processor and memory components described with respect to FIG. 9 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1000, or a portion thereof, constitutes a means for performing one or more steps of a method described herein.

In one embodiment, the chip set 1000 includes a communication mechanism such as a bus 1001 for passing information among the components of the chip set 1000. A processor 1003 has connectivity to the bus 1001 to execute instructions and process information stored in, for example, a memory 1005. The processor 1003 may include one or more processing cores with each core configured to perform independently. A multi-core processor enables multiprocessing within a single physical package. Examples of a multi-core processor include two, four, eight, or greater numbers of processing cores. Alternatively or in addition, the processor 1003 may include one or more microprocessors configured in tandem via the bus 1001 to enable independent execution of instructions, pipelining, and multithreading. The processor 1003 may also be accompanied with one or more specialized components to perform certain processing functions and tasks such as one or more digital signal processors (DSP) 1007, or one or more application-specific integrated circuits (ASIC) 1009. A DSP 1007 typically is configured to process real-world signals (e.g., sound) in real time independently of the processor 1003. Similarly, an ASIC 1009 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1003 and accompanying components have connectivity to the memory 1005 via the bus 1001. The memory 1005 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1005 also stores the data associated with or generated by the execution of one or more steps of the methods described herein.

### 5. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 6. REFERENCES

All references listed below and throughout the specification are hereby incorporated by reference in their entirety.
[1]. C. Lu and A. W. Czanderna, Applications of Piezoelectric Quartz Crystal Microbalances, Elsevier Science Publishers B. V., Amsterdam, N L, 1984.
[2]. G. Sauerbrey, Verwendung von Schwingquarzen zur Wägung dünner Schichten and zur Mikrowägung, Zeitschrift für Physik, 155 (2), 206-222 (1959).
[3]. W. G. Cady, Piezoelectricity: an introduction to the theory and applications of electromechanical phenomena, Dover Publications, New York, USA, 1964.
[4]. M. D. Ward and D. A. Buttry, In situ interfacial mass detection with piezoelectric transducers, Science, 249 (4972), 1000-1007 (1990).
[5]. F. Lack, G. Willard, and I. Fair, Some improvements in quartz crystal circuit elements, Bell System Technical Journal, 13 (3), 453-463 (1934).
[6]. D. A. Buttry and M. D. Ward, Measurement of interfacial processes at electrode surfaces with the electrochemical quartz crystal microbalance, Chemical Reviews, 92 (6), 1355-1379 (1992).
[7]. J. Janata, Principles of chemical sensors, Plenum Press, New York, USA, 1989.
[8]. gamry.com/application-notes/qcm/basics-of-a-quartz-crystal-microbalance/
[9]. biologic.net/topics/quartz-crystal-microbalance-measurement-principles/

What is claimed is:

1. An apparatus comprising:
(a) a crystal microbalance comprising an acentric, piezoelectric crystal material and directly evaporated onto a first side and a second opposite side, a pair of metal electrodes and
(b) a specific capture probe fixed directly or indirectly to the metal electrode on the first side of the crystal microbalance,
wherein the specific capture probe specifically binds to and captures a specific pathogen,
wherein capture of the pathogen causes a change in mass to the crystal microbalance, and
wherein the change in mass of the crystal microbalance causes a shift in a resonant frequency of the crystal microbalance.

2. The apparatus of claim 1, wherein the acentric piezoelectric material is a cut quartz crystal.

3. The apparatus of claim 1, wherein the metal is gold or a gold alloy.

4. The apparatus of claim 1, wherein the specific capture probe is selected from the group consisting of an ssDNA, a peptide, an aptamer, and a monoclonal antibody.

5. The apparatus of claim 4, wherein the specific capture probe is a monoclonal antibody.

6. The apparatus of claim 1, further comprising a capture membrane on top of the metal electrode and wherein the specific capture probes are attached on the capture membrane.

7. The apparatus of claim 1, wherein the specific pathogen is selected from the group consisting of SARS-CoV-2, influenza A, Bacillus anthracis, and Salmonella typhimurium.

8. The apparatus of claim 1, wherein the specific pathogen is SARS-CoV-2.

9. The apparatus of claim 1, further comprising a capture membrane situated adjacent to and in contact with the metal electrode on the first side of the crystal microbalance, and wherein the specific capture probe is fixed directly to the metal electrode on the first side of the crystal microbalance.

10. A system comprising:
(a) the crystal microbalance of claim 1;
(b) an oscillator connected to a pair of electrodes attached to opposite sides of a crystal of the crystal microbalance and configured to generate an alternating current (AC) voltage across the crystal to cause the crystal to vibrate at a resonant frequency;
(c) a frequency detection unit configured to measure a shift in the resonant frequency due to the change in the mass based on the specific capture probe binding to the pathogen in a medium directed at the crystal microbalance;
(d) at least one processor;
(e) at least one memory including one or more sequences of instructions, wherein the at least one memory and the one or more sequences of instructions are configured to, with the at least one processor, cause the system to perform at least the following;
(i) transmit a first signal to the oscillator to cause the oscillator to generate the AC voltage across the crystal,
(ii) receive a second signal from the frequency detection unit indicating a value of the shift in the resonant frequency due to the change in the mass,
(iii) determine a mass change on the crystal microbalance based on the shift in the resonant frequency,
(iv) relate the mass change to a pathogen load in the medium, and
(v) determine whether the pathogen load exceeds a high load threshold, and output a notification indicating an excessive load in the medium based on determining that the pathogen load exceeds the high load threshold.

11. The system of claim 10, further comprising a housing to mount the crystal microbalance, said housing including an inlet configured to direct the medium into the housing and an outlet configured to direct the medium out of the housing.

12. The system of claim 11, further comprising a fan positioned within the housing to direct the medium through the inlet toward the crystal microbalance.

13. The system of claim 11, further comprising a filter positioned within the housing between the inlet and the crystal microbalance, said filter configured to remove dust or debris from the medium such that the pathogen passes through the filter to the crystal microbalance.

14. The system of claim 11, wherein the housing comprises an electrostatic precipitator configured to direct the pathogen in the medium to the crystal microbalance.

15. The system of claim 10, wherein the oscillator is configured to generate the AC voltage to cause the crystal to vibrate over a range of frequencies including a first value of the resonant frequency and wherein the frequency detection unit is configured to measure an impedance across the electrodes over the range of frequencies and wherein the frequency detection unit is configured to determine the shift in the resonant frequency based on a difference between the first value of the resonant frequency and a second value of the resonant frequency within the range of frequency at which the measured impedance has a minimum value.

16. The system of claim 10, wherein the at least one memory and the sequences of instructions are configured to, with the at least one processor, cause the system to output the notification including to transmit a second signal to an output device to output an alert with the output device.

17. The system of claim 16, wherein the output device is one of a visual display configured to output a visual alert and an audible device configured to output an audible alert.

18. A method of detecting a pathogen in the ambient air of an enclosed space, comprising:
(a) placing the crystal microbalance of claim 1 into a sensor chip, calibrating a resonant frequency of the crystal microbalance of claim 1 to a mass on the crystal microbalance;
(b) conjugating the capture probe to the crystal microbalance and optionally blocking non-specific binding by exposing the conjugated surface to a blocking solution;
(c) installing the sensor chip in a housing and directing the ambient air or a sample of the ambient air at the sensor chip;
(d) measuring a resonant frequency of the crystal microbalance at predetermined time intervals and determining a mass change based on the measured resonant frequency at each predetermined time interval;
(e) relating the mass change to a pathogen load in the medium;
(f) determining whether the pathogen load in the ambient air exceeds a high load threshold; and
(g) outputting a notification indicating an excessive load in the ambient air based on determining that the pathogen load exceeds the high load threshold.

19. The method of claim 18, wherein the pathogen is SARS-CoV-2 virus particles.
